(19) United States
(12) Patent Application Publication
(10) Pub. No.: US 2024/0345191 A1
(43) Pub. Date: Oct. 17, 2024

(54) INTEGRATED B0-SHIM COIL CONFIGURATIONS FOR MRI B0 SHIMMING IN TARGET TISSUES

(71) Applicant: The Trustees of Columbia University in the City of New York, New York, NY (US)

(72) Inventors: M. Sebastian THEILENBERG, New York, NY (US); Carlotta IANNIELLO, New York, NY (US); Christoph JUCHEM, Ridgefield, CT (US)

(21) Appl. No.: 18/634,314

(22) Filed: Apr. 12, 2024

Related U.S. Application Data
(60) Provisional application No. 63/495,605, filed on Apr. 12, 2023.

(51) Int. Cl. G01R 33/3875; A61B 5/055; G01R 33/385

(52) U.S. Cl. CPC G01R 33/3875; A61B 5/055; G01R 33/385

(57) ABSTRACT

An apparatus for magnetic resonance imaging includes one or more radio-frequency (RF) loops configured to probe a measurement space and multiple coils configured for B0 shimming in the measurement space. The multiple coils are spaced such that interference with a received RF signal in the one or more RF loops is below a target level of interference.

### CROSS-REFERENCE TO RELATED APPLICATIONS

This application claims benefit of Provisional Application 63/495,605, filed Apr. 12, 2023, the entire contents of which are hereby incorporated by reference as if fully set forth herein, under 35 U.S.C. § 119(e).

### STATEMENT OF GOVERNMENTAL INTEREST

This invention was made with government support under Grant EB030560 awarded by the National Institutes of Health. The government has certain rights in the invention.

### BACKGROUND

The present invention relates to magnetic resonance imaging (MRI), magnetic resonance spectroscopy (MRS) and MRS imaging (MRSI), and in particular to magnetic resonance equipment suitable for an arbitrary image volume. In the following, the base magnetic field or static magnetic field may represent the combination of the external magnetic field and the macroscopic effects of the tissue susceptibility.

Nuclear magnetic resonance studies magnetic nuclei by aligning them with an applied constant magnetic field (B0) in direction z and perturbing this alignment using an alternating magnetic field (B1) at radio frequencies (called RF pulses), orthogonal to z. The resulting response to the perturbing magnetic field is the phenomenon that is exploited in magnetic resonance spectroscopy (MRS) and magnetic resonance imaging (MRI). Spatial distributions of the measured spectroscopy are determined by adding to B0, the strength in the z direction, a spatial gradient of the z direction magnetic field along each of three orthogonal coordinate dimensions (e.g., x, y and z) designated Gx, Gy, Gz, respectively.

During data acquisition, the most commonly employed strategy for improving spectral resolution is the automated technique for improving the homogeneity of the magnetic field B0. Fast and high order shimming techniques have been implemented on modern scanners using spherical harmonics (SH), typically up to second order, to make B0 more uniform across a subject being scanned, yet these methods cannot eliminate all variation in local magnetic fields that are caused by the differing magnetic susceptibilities of various interposed tissues within the body and air inside and surrounding the body. For example, in imaging certain organs, such as the brain and the heart, the susceptibility differences between adjacent substances, such as the air in the sinuses and the ear canals and the bones of the skull, jaw and teeth from the brain tissue, or air within the lungs from the heart muscle tissue, create strong local inhomogeneities, e.g., in the prefrontal cortex and medial temporal lobes, or heat respectively.

### SUMMARY

Techniques are provided for improving magnetic field generation in MRI, MRS and MRSI applications among other applications using one or more B0-shim-only coils (also called multi-coils, MC, herein for legacy reasons and because they are often used in multiples) for B0 shimming only. The MC techniques, which uses a set of one or more non-specific small coils driven individually, outperforms SH shimming. While early MC setups used dedicated coils that need additional space and can affect signal to noise ratio (SNR), especially when placed inside the RF coil, some MC hybrid setups used the RF coil elements for MC shimming as well, to minimize space requirements. However, since RF loops consist of only one turn of wire with limited maximum DC current, and, moreover, since the RF coil geometry is not optimized for B0 control, the shim capabilities of such a system are reduced compared with dedicated MC-only systems. In this work, both approaches are integrated by adding dedicated MC shim elements to a shim-capable RF coil, adapting the size, location, and number of MC elements via simulations based on in vivo tissue B maps to minimize interactions, especially minimize MC-to-RF coupling due to mutual inductance.

In a first set of embodiments, an apparatus for magnetic resonance imaging includes one or more radio-frequency (RF) loops configured for probing a measurement space. The apparatus also includes multiple coils configured only for B0 shimming in the measurement space. The multiple coils are spaced such that interference with a received RF signal in the one or more RF loops is below a target level of interference.

In some embodiments of the first set, B0 is directed in a z direction, and the one or more RF loops are centered at z position z_{L}. N is a number of rows of the one or more RF loops in the z direction. Each loop has a diameter no smaller than d_{L}. Each coil of the multiple coils has a diameter no larger than a maximum diameter d_{C} that is smaller than N d_{L} The multiple coils are arranged in at least two sets of rows, a first set centered at a z position z1 less than z_{L} and a second set centered at a z position z2 greater than z_{L}. Each set of rows is on a periphery of the measurement space. A distance [symbol] z=z2−z1, and d_{C}< [symbol] z<N d_{L}.

In some embodiments of the first set, the measurement space is a cylinder. In some embodiments of the first set, each loop is configured to carry up to 1 ampere of current independently of every other loop and each coil is configured to carry at least the current of each loop. In some embodiments of the first set, each loop has only one winding of an insulated conductor and each coil has at least 10 windings of an insulated conductor. In some of these embodiments, each coil has 20 to 80 windings. In some of the embodiments, each coil has about 50 windings.

In other embodiments, an MRI, MRS or MRSI system, or a method, or a computer-readable medium, or computer system is configured to use the above apparatus.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is a block diagram that illustrates an example MR system, according to an embodiment;

FIG. 2A is a block diagram that illustrates an example cross section of a MR system in a x-y plane perpendicular to the direction z of the magnetic field B0, according to prior art;

FIG. 2B through FIG. 2D, are block diagrams that illustrate example coils distributed around the bore hole of FIG. 2A, which can serve as x-gradient, y-gradient and z gradient coils in the z-directed magnetic field, according to prior art;

FIG. 3 is a perspective drawing that illustrates an example individual coil unit for shimming only, according to an embodiment;

FIG. 4 is a block diagram that illustrates example MR RF loops for a cylindrical measurement space, according to an embodiment;

FIG. 5A through FIG. 5E are block diagrams that illustrate example spatial positions for two sets of rows of B0 shimming only MC for use with the RF loops of FIG. 4, according to various embodiments;

FIG. 6 is a block diagram that illustrates three example spacings of MC in each set of rows, for the three sets of rows depicted in FIG. 5A, FIG. 5C and FIG. 5E around the RF loops of FIG. 4, according to various embodiments.

FIG. 7A and FIG. 7B are block diagram that illustrates example parameters that describe the positions of B0 shimming only MC in two sets of rows, according to an embodiments.

FIG. 8A through FIG. 8C are plots that illustrate example Shim performance for different spatial arrangements of B0 shimming only MCs, according to various embodiments;

FIG. 9A through FIG. 9E are block diagrams that illustrate example spatial arrangements of RF loops and MC for the best performing simulated setup for each basic geometry, according to an embodiment;

FIG. 10 is a plot that illustrates example shim performance of the configurations of FIG. 9A through FIG. 9E compared to traditional spherical harmonics (SH) shimming, according to various embodiments;

FIG. 11A through FIG. 11F are images that illustrate simulated shimmed B0 field distribution in one central axial slice of one subject for six different configurations of B0 shimming only MCs, according to various embodiments;

FIG. 12A and FIG. 12B are block diagrams that illustrate the simulation setup to test the effect on signal to noise ratio (SNR) of MC-to-RF coupling due to mutual inductance, according to an embodiment;

FIG. 13A through FIG. 13D are images that illustrate example simulated SNR maps in spherical phantom, according to an embodiment;

FIG. 14A through FIG. 14C are photographs that illustrate example Q factor measurements of a single loop, according to various embodiments;

FIG. 15 is a table that illustrates example results from Q factor measurements that indicate a MC coil contributes less than 25% of the noise at the RF loop, according to an embodiment;

FIG. 16A through FIG. 16D are cylindrical maps that illustrate example magnetic flux obtained from sampling the array magnetic field flux through three cylindrical surfaces at different radii, according to an embodiment;

FIG. 17 is a method for configuring an apparatus with RF loops and B0 shim-only MC coils, according to an embodiment;

FIG. 18A is a block diagram that illustrates an example of a spherical phantom for a human head inside an array of RF loops, according to an embodiment; and FIG. 18B is a block diagram that illustrates the MR RF loops for a helmet-like measurement space, according to an embodiment.

FIG. 19A, FIG. 19B and FIG. 19C are greyscale images that illustrate examples of simulated signal-to-noise ratio (SNR) in example axial, coronal and sagittal slices, respectively, from the RF coil setup illustrated in FIG. 18 before addition of MC shim only coils, according to an embodiment of the RF loops;

FIG. 19D is an greyscale image of simulated magnetic flux generated by the 16-channel array through an elliptical cylinder distancing 30 mm from the RF coil, according to an embodiment of the RF loops;

FIG. 20 is a block diagram that illustrates an example of a flattened 2D circuit schematic of an RF coil, according to an embodiment of the RF loops;

FIG. 21A, FIG. 21B and FIG. 21C are greyscale images that illustrate examples of transverse, sagittal and coronal slices, respectively, of measured SNR of the 16-channel RF array before inserting the RF chokes or the MC shim-only coils, according to an embodiment of the RF loops;

FIG. 21D is a set of greyscale images that illustrate examples of measured coil sensitivity for each loop of the RF array in its RF-only configuration, according to this embodiment of the RF loops;

FIG. 21E is plot showing example locations for MC coils superposed on the greyscale image of the simulated magnetic flux of FIG. 19D, according to an embodiment;

FIG. 22 is a block diagram that illustrates an example schematic representation of MC coils (4 rows of 8 circular elements) surrounding the RF coil (2 rows of 8 squared loops, appropriately bent to follow the contour of a head helmet), according to an embodiment;

FIG. 23A is a photograph that depicts an example of a multicoil shim-only coil element, according to an embodiment.

FIG. 23B is a photograph that depicts an example panel of multiple shim-only coils, according to an embodiment.

FIG. 23C is a block diagram that illustrates the experimental panel 2340 with MC shim-only coils 2320 placed adjacent a helmet of RF elements (loops) 2310, according to an embodiment.

FIG. 23D is a photograph that illustrates the experimental panel 2340 with MC shim-only coils 2320 placed adjacent a helmet of RF elements (loops) 2310, according to an embodiment

FIG. 24A, FIG. 24B and FIG. 24 C are greyscale images that illustrated examples of B0 field maps produced by MC shim-only coils 2320a, 2320b and 2320c, respectively, depicted in FIG. 23B, according to an embodiment; and FIG. 24 D is a high resolution MR image of the spherical phantom used for the experiment, according to an embodiment.

FIG. 25 is a block diagram that illustrates a computer system upon which an embodiment of the invention may be implemented; and

FIG. 26 illustrates a chip set upon which an embodiment of the invention may be implemented.

### DETAILED DESCRIPTION

In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of imaging the human head. However, the invention is not limited to this context. In other embodiments, B0 shim-only coil units described herein are used with one or more RF-only or RF-plus-shim loops in different assemblies. e.g., assemblies to image other arbitrary spaces, one-sided open or enclosed, such as suitable for a particular organ or hand or foot or non-human animal or manufactured part.

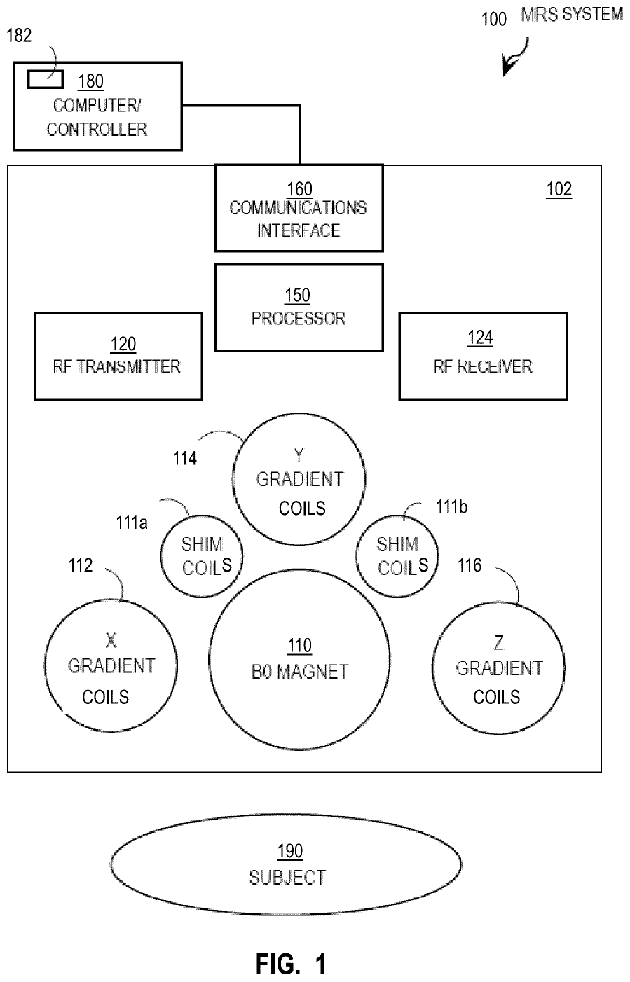

In various embodiments, B0-shim-only coils or steps of the method described below are incorporated into an MR scanning system or apparatus or measurement. FIG. 1 is a block diagram that illustrates an example MR system 100, according to an embodiment. The system 100 includes a scanning apparatus 102 and a computer/controller 180. Although a subject 190 for MR scanning is depicted in FIG. 1, subject 190 is not part of the MR system 100.

The scanning apparatus 102 includes a B0 magnet 110, an x-gradient magnet 112, a y-gradient magnet 114, a z-gradient magnet 116, a group of shim coils (e.g., shim coils 111a and 111b), a radio frequency (RF) transmitter 120, an RF receiver 124, a communications interface 160, and a processor 150. In various embodiments, the coil units described herein each, or in some combination, replace in whole or in part one or more of coils 111a or 111b or both.

The computer/controller 180 sends signals to the communications interface 160 in scanner 102 that causes the scanner to operate the shim magnets 111a and 111b and the gradient magnets 112, 114 and 116 and the RF transmitter 120 and RF receiver 124 to obtain MRI or MRS or MRSI data.

In the illustrated embodiment, computer/controller 180 includes module 182 to drive a multi-coil (MC) subsystem and process MR data produced as a result. Module 182 performs one or more steps of the method described below for operating gradient and shim coils in the course of imaging a target object, such as the human head, other organ, non-human animal, or tissue, or manufactured part. The collected data is received at computer/controller 180 or another computer and used to present or use a MR measurement.

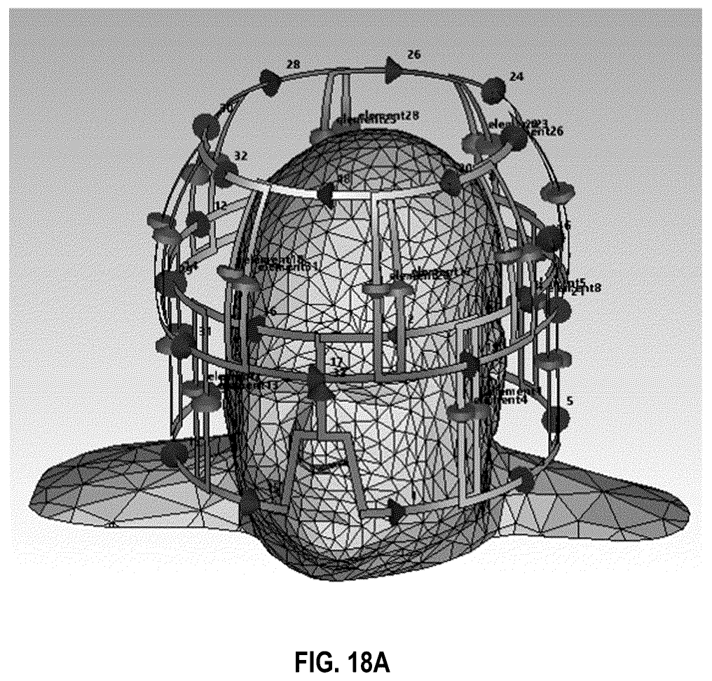

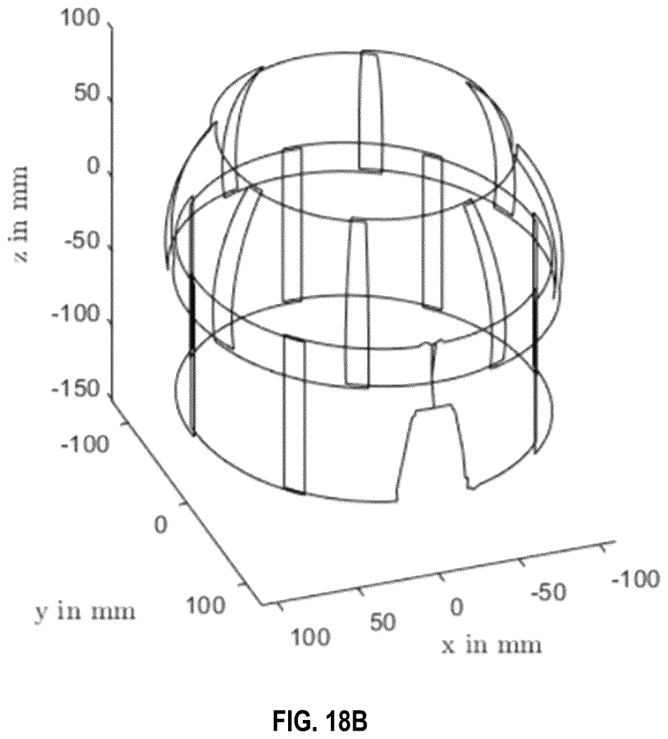

In some embodiments, module 182 is included in processor 150 within scanner 102. In various embodiments, one or more steps of the method are performed by multiple processors on scanner 102 or computer/controller 180 or other computers connected to computer/controller 180 via a network, as depicted in FIG. 18.

Although processes, equipment, and data structures are depicted in FIG. 1 as integral blocks in a particular arrangement for purposes of illustration, in other embodiments one or more processes or data structures, or portions thereof, are arranged in a different manner, on the same or different hosts, in one or more databases, or are omitted, or one or more different processes or data structures are included on the same or different hosts.

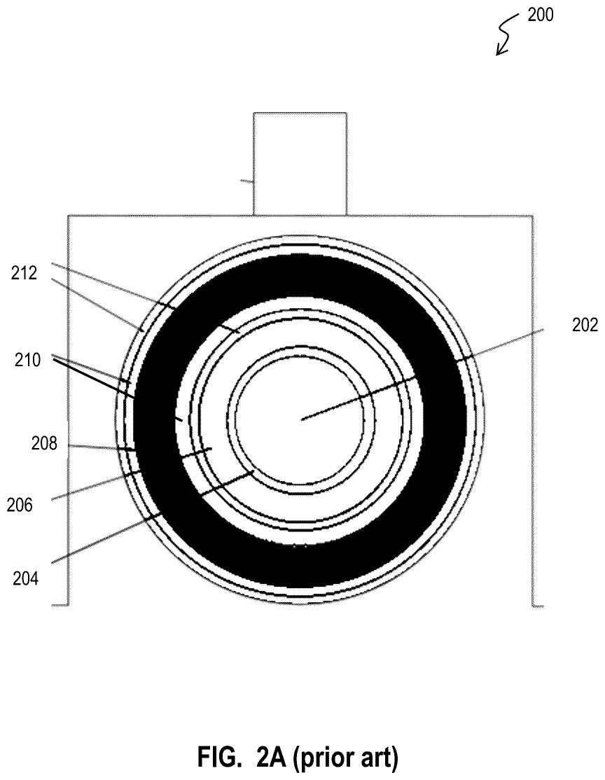

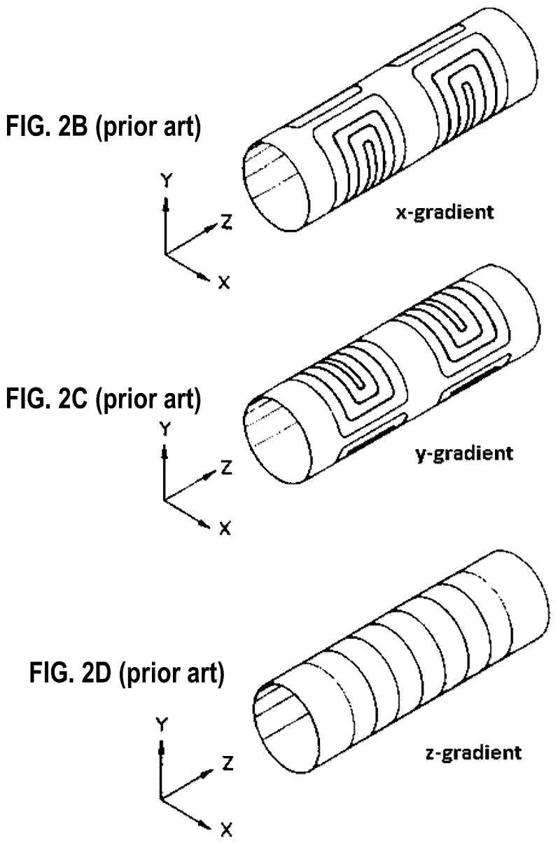

FIG. 2A is a block diagram that illustrates an example cross section of a prior art MR measurement system 200 in a x-y plane perpendicular to the direction z of the magnetic field B0, upon which an embodiment may be implemented. The system 200 is roughly cylindrical with the z-direction field B0 oriented along the rotational axis of symmetry for the cylinder. The scanner bore 202 is a cylindrical space into which a subject is inserted. Successively outward layers include RF transmit coil 204, the magnetic gradient coils 206, a superconducting primary electromagnetic coil 208 to provide the B₀ field between two cooling layers 210, e.g., filled with liquid helium, to cool the primary magnet to superconducting temperatures, and thermal insulation layers 212.

FIG. 2B through FIG. 2D, are block diagrams that illustrate example prior art coils distributed around the bore hole of FIG. 2A, which can serve as x-gradient, y-gradient and z gradient coils in the z-directed magnetic field, according to an embodiment. These coils occupy layer 206 in FIG. 2A. These coils are operated to determine the spatial distribution of the MR measurements during imaging.

### 1. Overview

As described herein, after initial work on multi-coils, it was recognized separate shim-only multi-coils can be deployed with placement strategies that minimize interference with RF coils and thus minimize the consequent SNR penalty. It is shown that this approach overcomes the challenging B0 inhomogeneities in difficult tissues such as the frontal brain and animal heart.

A target level of interference can be defined in any number of ways. For example, an increase of SNR of less than 50%, or less than 30%, or less than 10%, compared to the SNR before introduction of the B0-shim-only coils, is a target level of interference in various embodiments.

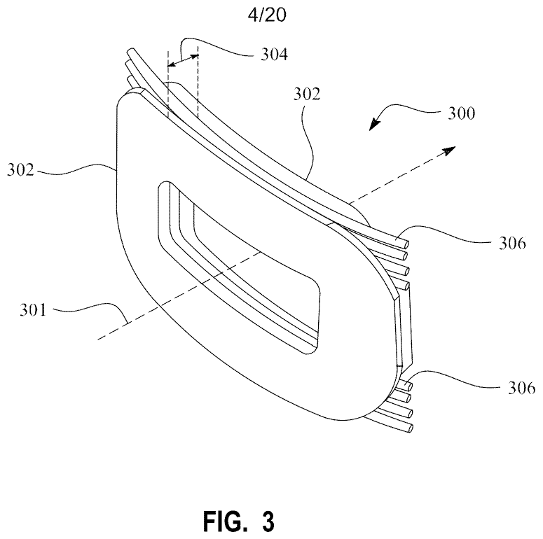

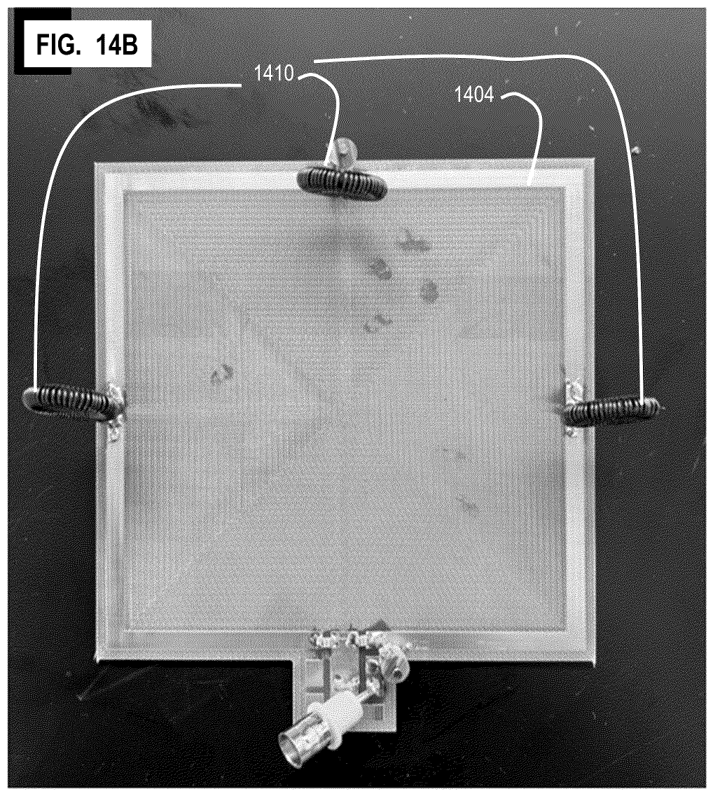

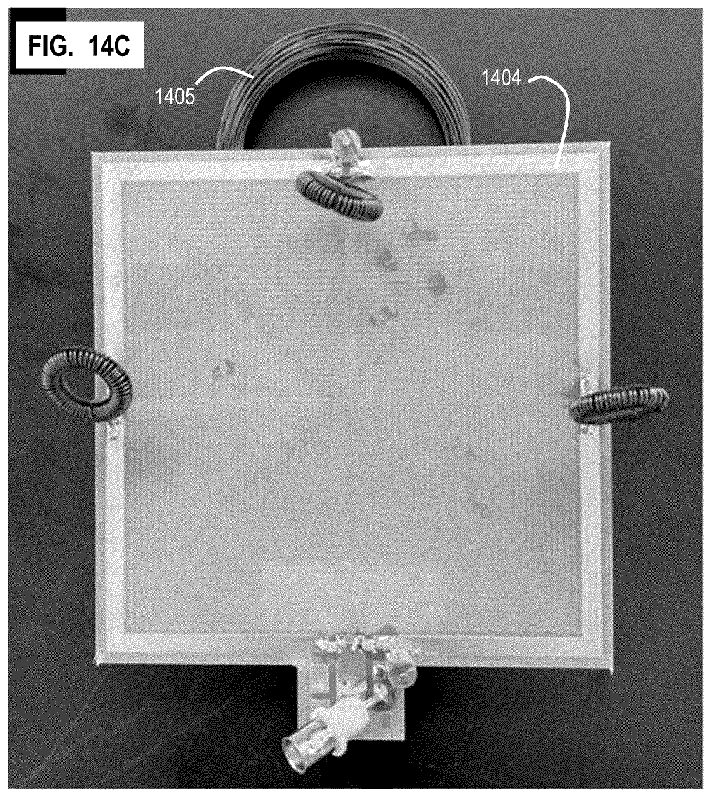

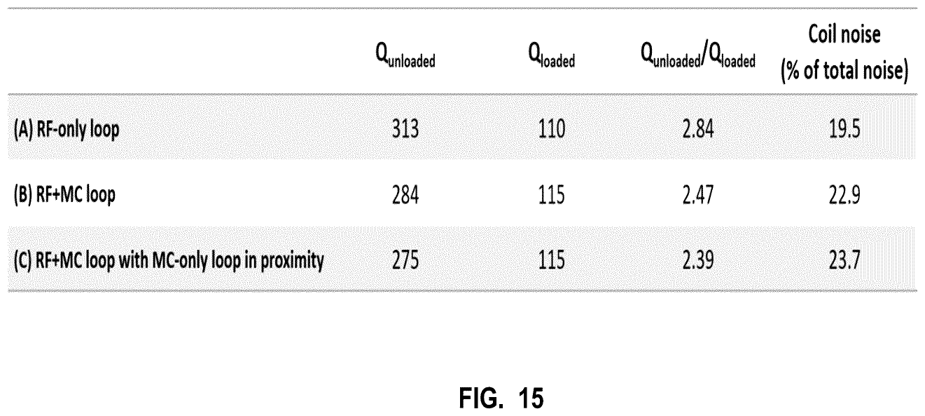

FIG. 3 is a perspective drawing that illustrates an example individual coil unit 300, according to an embodiment. In other embodiments, several features depicted in FIG. 3 are changed or omitted for a particular purpose, e.g., to reduce size or power usage. See, for example, the simple coil 1405 of multiple windings in FIG. 14C. The illustrated individual coil unit 300 is designed as a sandwich of two coil elements 302, each called a winding hereinafter, each winding composed of one or more wire loops. In some embodiments, a coil unit comprises more or fewer than two such coil elements. The windings are electrically connected in series. To avoid short circuits the wire is electrically insulated. Note that the windings 302 define a longitudinal axis 301 approximately perpendicular to the loops in each winding. Note also that in the illustrated embodiment, the windings 302 are non-planar to conform to a curved shell that will be constructed by assembling multiple such coil units to enclose, at least in part, a MR measurement space. This means the windings 302 are not exactly perpendicular to the axis 301. However, each winding is configured to provide a circulating current that induces a spatially specific localized magnetic field. Thus, one pair of leads is used to send an electric current through all windings 302 of a single coil unit 300.

A gap 304 in the longitudinal direction, parallel to the longitudinal axis 301, is included between the two sandwiching elements 302; and, is used for placement of cooling tubes 306 for cooling of the coil elements 302. In other embodiments, for reduced space, the double windings of FIG. 3 are replaced by a single winding and the gap 304 and cooling tubes 306 are omitted, as air cooling is sufficient.

### 1.1 Magnetic Resonance Apparatus

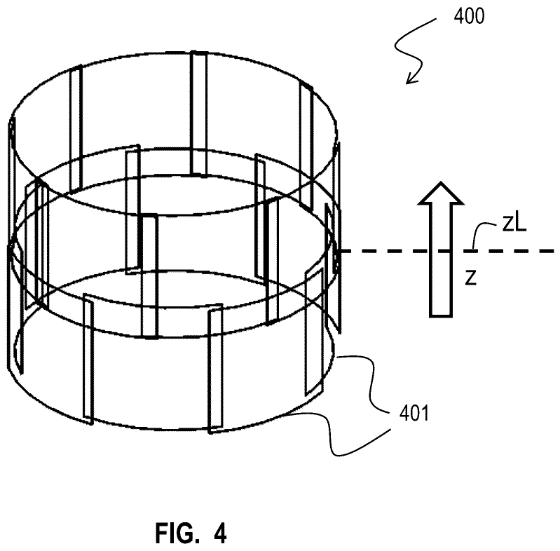

FIG. 4 is a block diagram that illustrates example MR RF loops 401 for a cylindrical measurement space, according to an embodiment. Each loop 401 is a single winding with diameter d_{L} of an insulated conductor that can have a current flow through separately from any other loop. Each can also maintain a DC current in a range from about −1 ampere to about +1 amperes. In practice, the RF loops we are limited by the current rating of the lumped elements (e.g., inductors, chokes) and currents in the range of +/−1 A is common. Specifically, increasing the max current would require bulkier RF chokes, which would take up more space and potentially get in the way of other components. The collection of RF loops 401 is called the RF array 400. Though depicted as overlapping arced rectangular RF loops 401 enclosing a cylindrical measurement space, in other embodiments, other shapes for the same number or more or fewer RF loops 401 and other shapes for the measurement space are used. In general, an RF array is configured to probe the RF signals in a measurement space. In the illustrated embodiment, the B0 field direction, also called simply the z direction, is in the direction of the axis of the cylindrical space. For simplicity of explanation the z coordinate is assumed to increase upward in the illustrated cylinders. The array 400 is centered in the z direction at z position z_{L}. The array 400 includes two rows of loops 401 when traversing the array in the z direction. In general, one or more RF loops includes N rows in the z direction, where N≥1. Thus, the size of the array 400 in the z direction is N d_{L}.

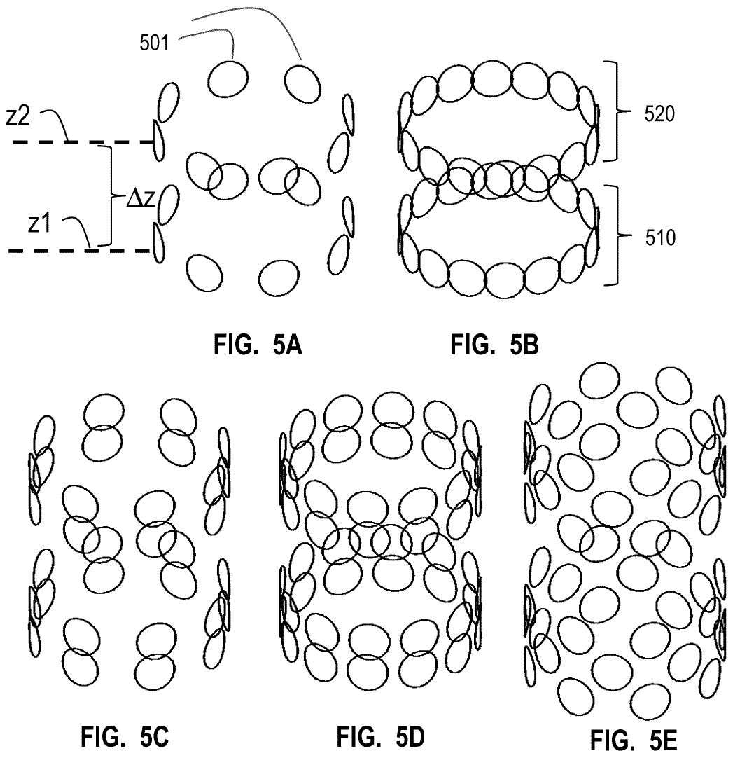

FIG. 5A through FIG. 5E are block diagrams that illustrate example spatial positions for two sets of rows of B0 shim-only MC for use with the RF loops of FIG. 4, according to various embodiments. In each figure the z direction is upward, as in FIG. 4. Each circle in FIG. 5A through FIG. 5E represents one coil 501 of the B0 shim-only MCs and each coil 501 includes many more windings than the RF loop—each coil having on the order of 10 to 100 windings. The number of turns is directly related to the maximum current and desired field strength. For shimming purposes, ranges are 10 to 50 turns with 1 A current, 20 to 100 turns with 0.5 A current, and 50 to 200 turns with 0.2 A current, according to various embodiments. The coils 501 are arranged on the periphery of the measurement space, and thus mime the cylindrical measurement space enclosed by the loop array 400.

As described in more detail below, it is advantageous for the MCs to be arranged apart from z_{L} to reduce cross inductance and the resulting interference. Thus, in each arrangement, the MCs 501 are arranged into two sets of rows, 510 and 520, one set 510 centered in the z direction below z_{L} at z1, and one set 520 centered in the z direction above z_{L} at z2. The difference, z2−z1= [symbol] z is a distance that serves as a parameter of the spatial positioning of the MCs 501. The difference [symbol] z tends to be less than the size of the RF loop array in the z direction, e.g., [symbol] z<N d_{L}, in some embodiments.

Each set 510, 520 of rows may have one or more rows of MCs. In FIG. 5A and FIG. 5B there is one row of MCs 501 in each set. In FIG. 5C and FIG. 5D there are two rows in each set. In FIG. 5E there are three rows in each set. In the illustrated embodiments, there is the same number of rows in each set. In some embodiments, there is a different number of rows in the two sets 510 and 520. Each row may have any number of coils. In the illustrated embodiment, there is no overlapping of coils 501 in a row so the number of coils 510 is limited by the diameter of each coil, d_{C} and the circumference of the periphery of the measurement space. In FIG. 5A, FIG. 5C and FIG. 5E there are 8 coils 501 in each row. In FIG. 5B there are 16 coils 501 in each row. In FIG. 5D there appear to be 12 coils 501 in each row.

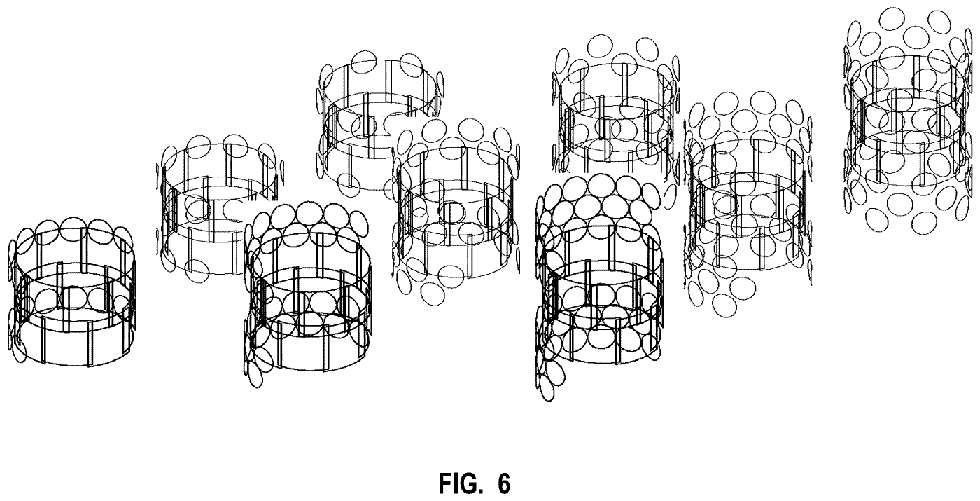

The coils 501 need not be evenly distributed around the periphery of the measurement space. In some embodiments, the MCs 501 are bunched toward one side, called the front for convenience, because they would be closest to the tissue of interest, such as the frontal cortex of the brain. FIG. 6 is a block diagram that illustrates three example spacings of MCs 501 in each set of rows, for the three sets of rows depicted in FIG. 5A, FIG. 5C and FIG. 5E around the RF loops 401 of FIG. 4, according to various embodiments. A parameter of the positioning that captures these types of variation is the angle [symbol] between coils in a row. The smaller [symbol] , the more bunched the coils 501 are on one side of a row. Three values of [symbol] are shown for each set of rows.

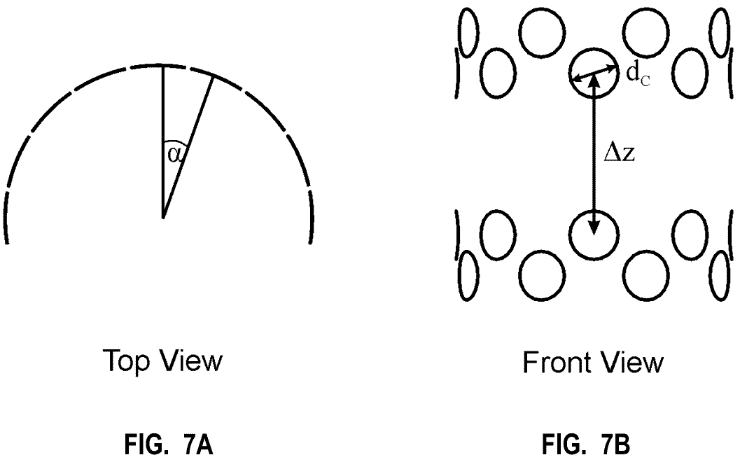

FIG. 7A and FIG. 7B are block diagram that illustrates example parameters that describe the positions of B0 shimming only MC in two sets of rows, according to an embodiment. FIG. 7A shows the angle (between centers of adjacent coils, when [symbol] is relatively small so all coils 501 are bunched on one side of the row. FIG. 7B is a side view, also called front view, that depicts the diameter d_{C} of each coil 501 and the distance [symbol] z between the two sets of rows.

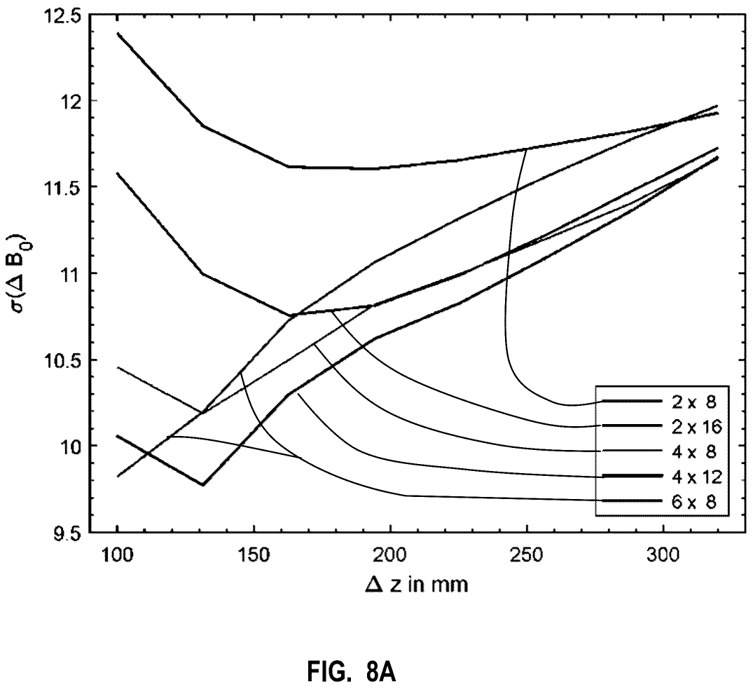

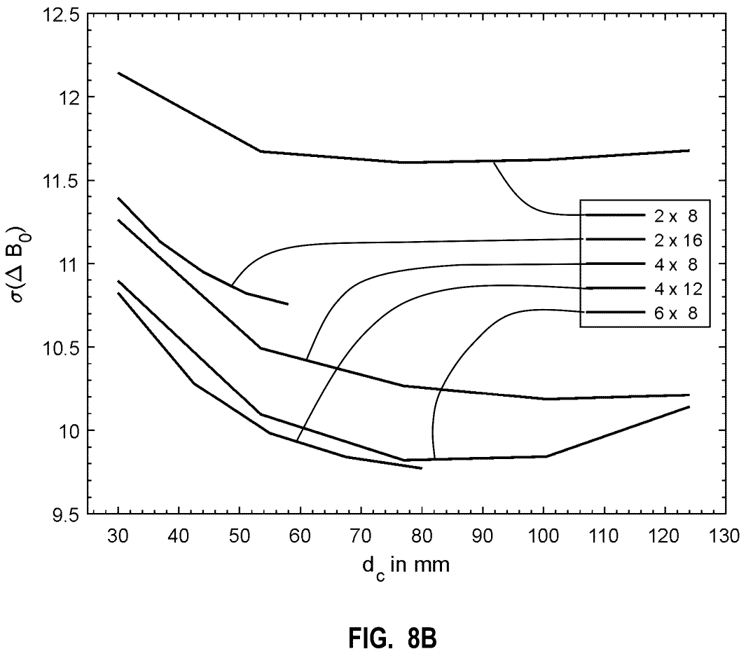

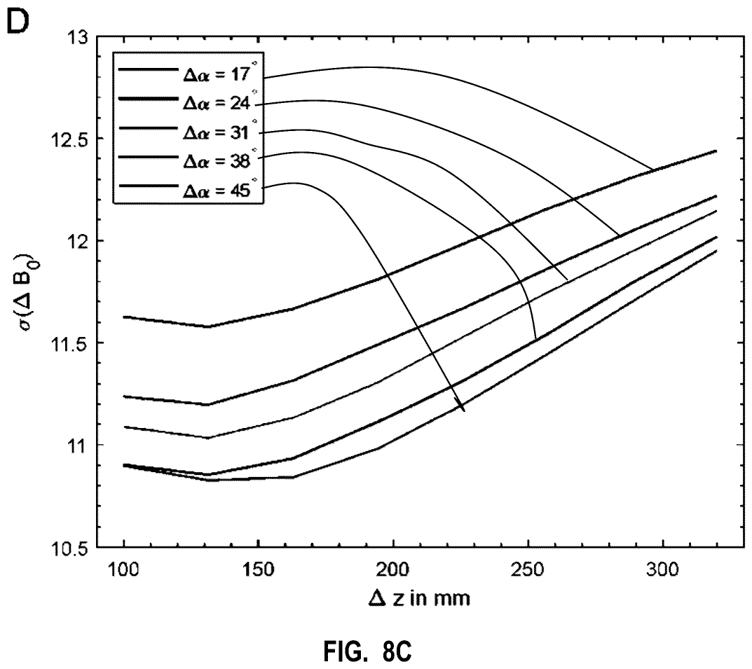

FIG. 8A through FIG. 8C are plots that illustrate example shim performance for different spatial arrangements of B0 shim-only MCs, according to various embodiments. Shim performance is measured by the variance in the values of B0 across the tissue of interest, designated [symbol] [symbol] B0). The higher the variance the less desirable the performance. These variances were simulated for brain tissue using the methods described below. The legend indicate the number of rows in both sets 510 and 520 totaled by the number of coils 501 per row. In FIG. 8A and FIG. 8B, the coils are equally spaced along the row. In FIG. 8A the variance is plotted against the separation distance [symbol] z. Here the arrangement with the fewest coils (2×8=16) does the worst. The best is 4 rows of 12 coils=48 coils. The same number of coils but spread over 6 rows (6×8) does not as well. The best performance for any number of coils, tends to be at values of [symbol] z about 100 millimeters (mm, 1 mm=10₋₃ meters). In FIG. 8B the variance is plotted against the coil diameter d_{C}. Here the performance improves with increasing diameter to a diameter of about 80 mm, after which there is little improvement. Note that the diameter of the RF loop array, N d_{L}, is about 240 mm, so d_{C}< [symbol] z<N d_{L} where performance is good. In FIG. 8C the coils are not all equally spaced along the row. These arrangements are for 4 rows with 8 coils each and each coil having diameter d_{C}=40 mm for various α (called [symbol] α in the plot). These show the best performance when evenly spaced, in this case [symbol] =45°.

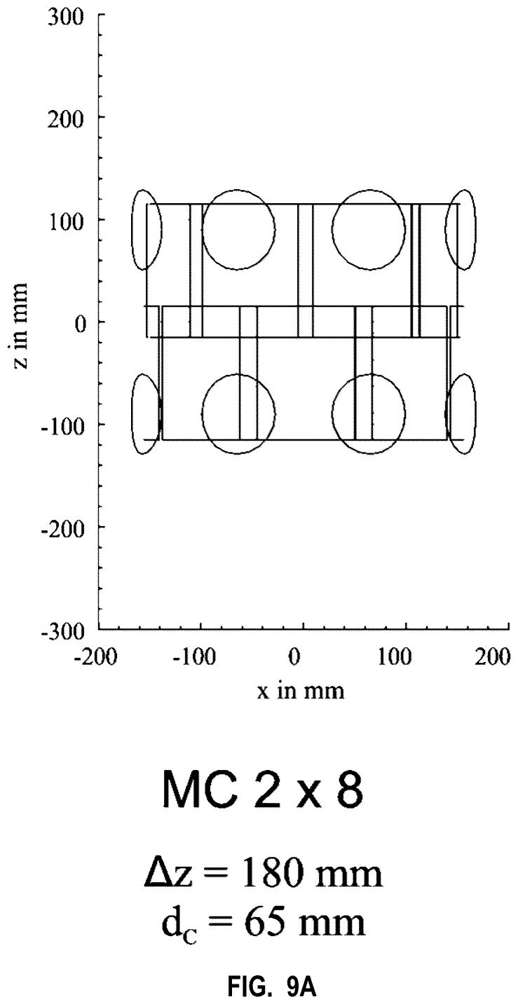

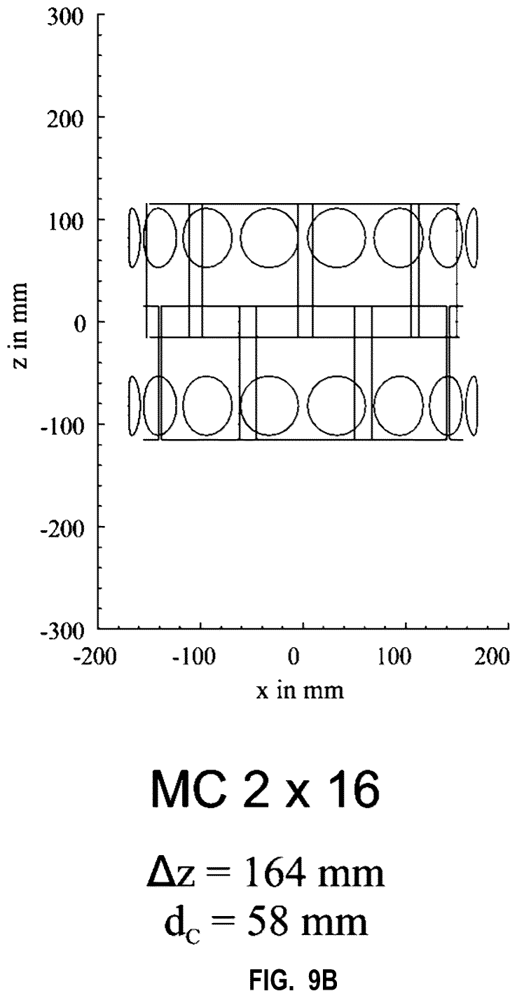

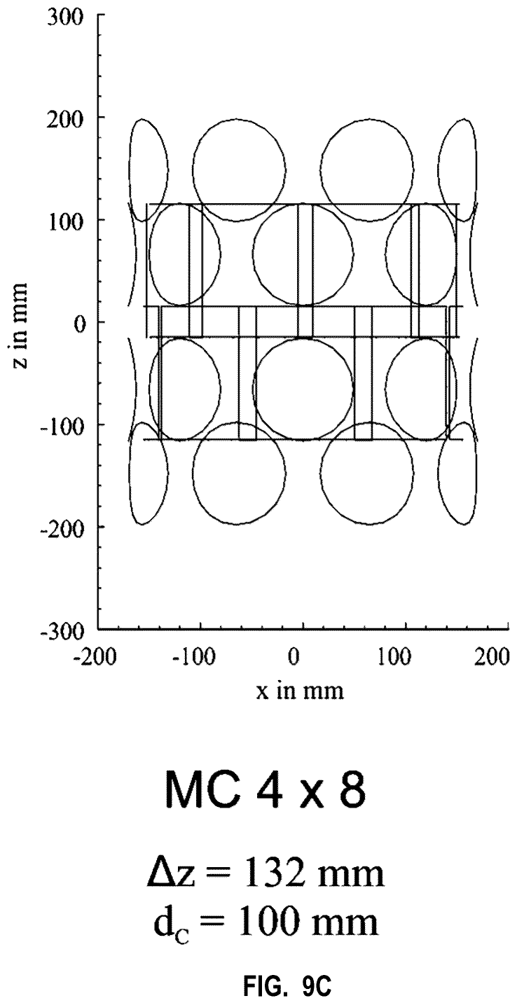

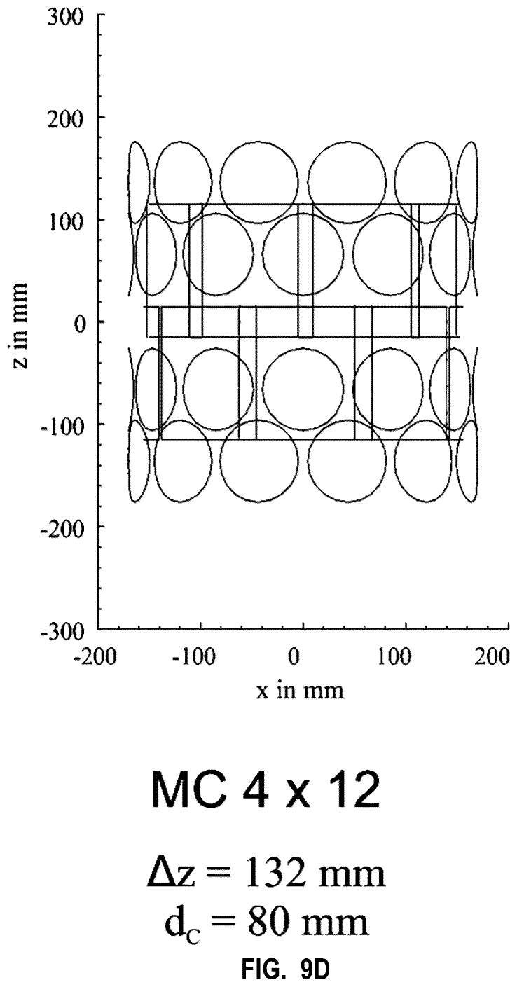

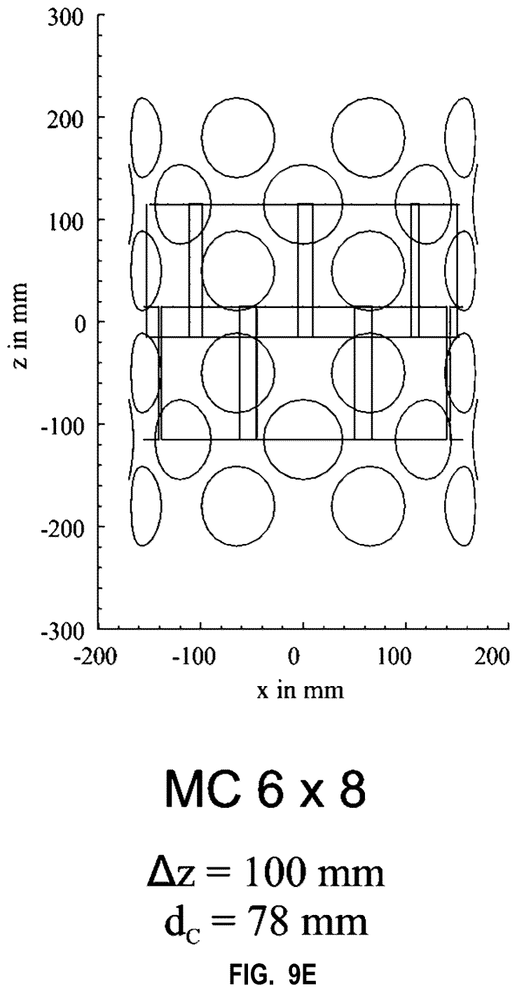

FIG. 9A through FIG. 9E are block diagrams that illustrate example spatial arrangements of RF loops and B0 shim-only MC for the best performing simulated setup for each basic geometry, according to an embodiment. Here the basic geometries are the same as plotted in FIG. 8A. The spacing parameter values given on each plot provide the lowest variance in B0 across the simulated tissue.

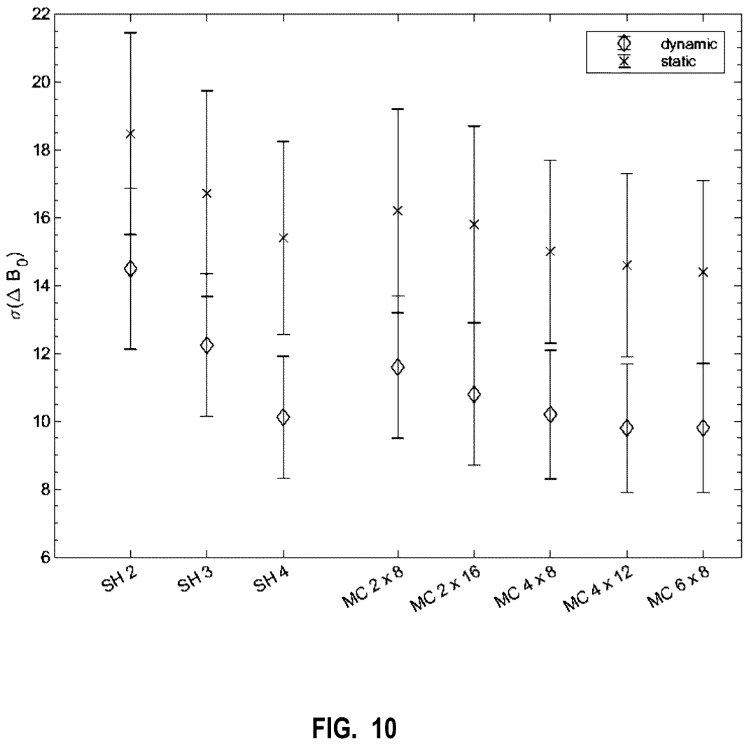

FIG. 10 is a plot that illustrates example shim performance of the configurations of FIG. 9A through FIG. 9E compared to traditional spherical harmonics (SH) shimming, according to various embodiments. Shim performance of the optimal setups for each basic geometry compared to traditional spherical harmonics (SH) shimming, both in static global shimming of the full brain ROI, and in dynamic slice-by-slice shimming. Shown is the average shim performance σ(ΔB) and its standard deviation across all 139 in vivo subjects. Note that dynamic shimming above 1st order is currently not available in clinical systems. For all the optimized geometries the addition of the B0 shim-only MC outperforms spherical harmonics currently available clinically.

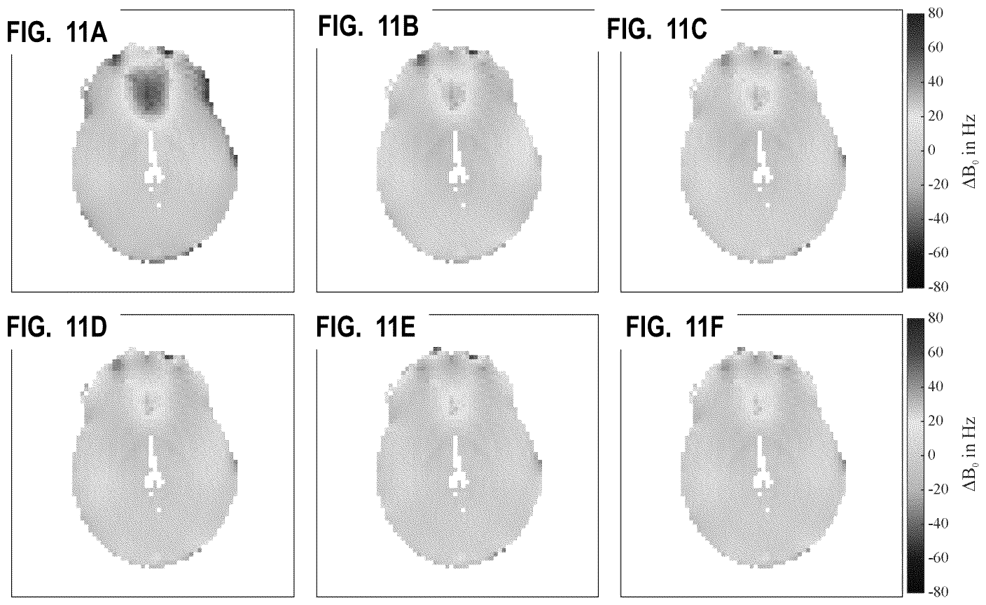

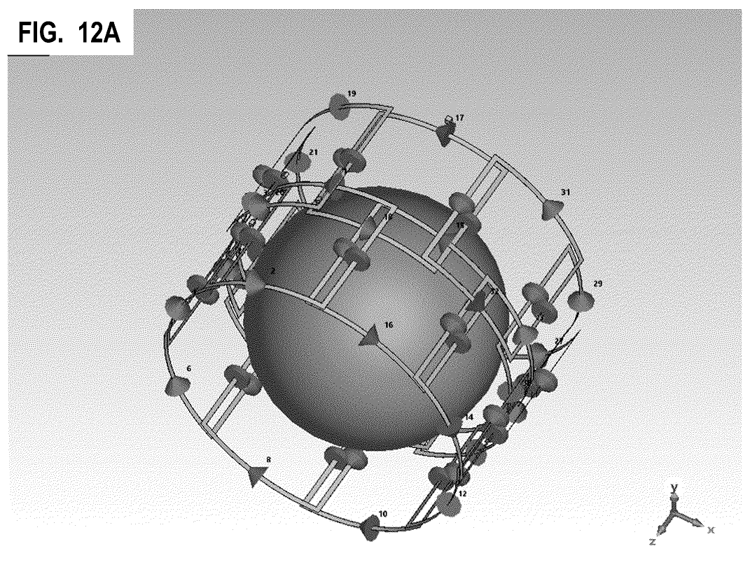

FIG. 11A through FIG. 11F are images that illustrate simulated shimmed B0 field distribution in one central axial slice of one subject for six different configurations of B0 shim-only MCs, according to various embodiments. FIG. 11A shows that after 2nd order static SH shimming, as is the clinical standard today, there is excessive B0 field strength in the central dark spot and deficient B0 field strength adjacent the skull. After dynamic multi-coil shimming using the best calculated setup, FIG. 11B shows the results with 2×8 coils, FIG. 11C shows the results with 2×16 coils, FIG. 11D shows the results with 4×8 coils, FIG. 11E shows the results with 4×12 coils, and FIG. 11B shows the results with 6×8 coils. All are clearly superior to clinical spherical harmonic shimming, with well reduced excessive field strength and no significant deficiencies shown in FIG. 11A.

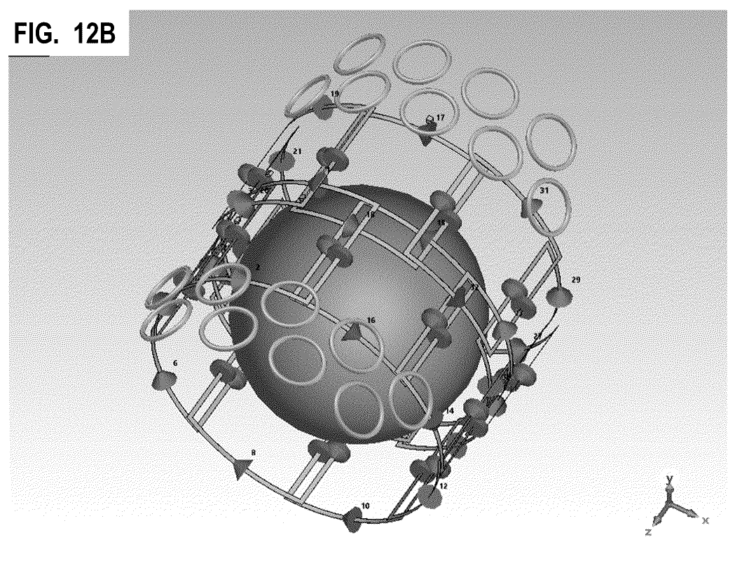

FIG. 12A and FIG. 12B are block diagrams that illustrate the simulation setup to test the effect on signal to noise ratio (SNR) of MC-to-RF coupling due to mutual inductance, according to an embodiment. FIG. 12A depicts a 16 channel RF-only receive array for a human head. FIG. 12B depicts a 16-ch RF-only with 18 B0 shim-only MC coils (radius 25 mm) distributed over the frontal portion of the coil above and below the RF-only array. Note that the two sets of B0 shim-only coils (MC elements) are centered above and below all the loops of the RF array. A spherical phantom with average dielectric properties in brain white and gray matter at 123 MHz was used to load the coil. This choice corresponds to a B0 field strength of 3 Tesla (T). In other embodiments, other field strengths for B0 are used.

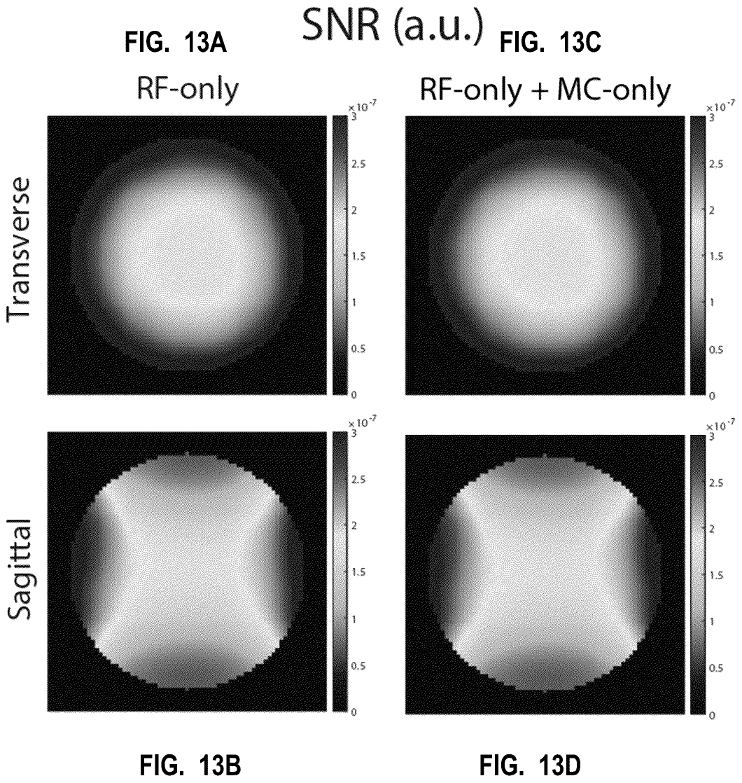

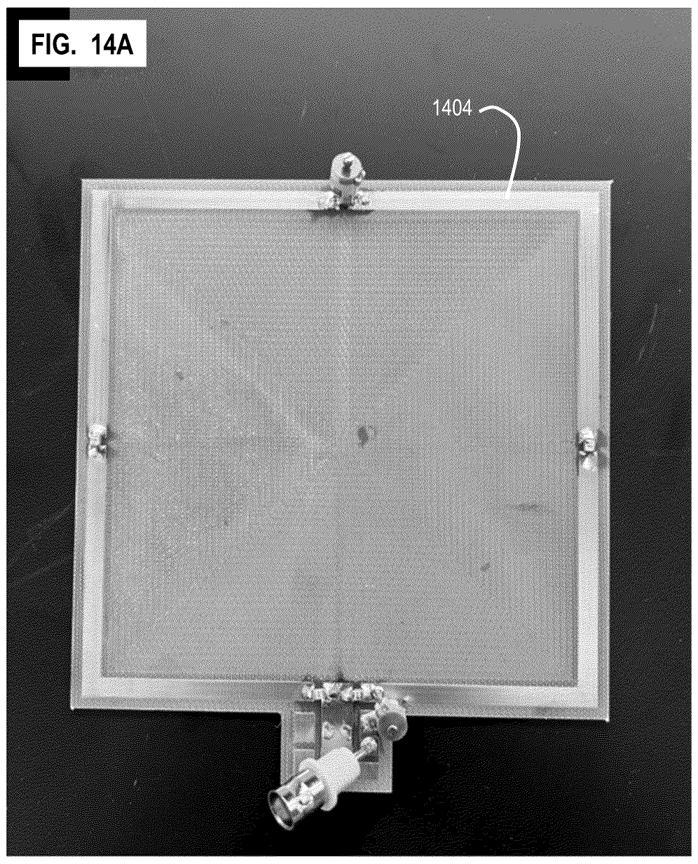

FIG. 13A through FIG. 13D are images that illustrate example simulated SNR maps in spherical phantom, according to an embodiment. FIG. 13 A and FIG. 13C are traverse sections perpendicular to the body axis of the subject dividing up from down. FIG. 13 A and FIG. 13C are sagittal sections parallel to the body axis of the subject and dividing left from right. The presence of the B0 shim-only MC elements has negligible impact on SNR.

FIG. 14A through FIG. 14C are photographs that illustrate example Q factor measurements of a single loop, according to various embodiments. The single RF loop is the square perimeter conductor 130 mm on each side bordering the pegboard surface. FIG. 14A depicts the RF loop 1404 only. FIG. 14B depicts the RF loop with RF chokes 1410. RF chokes are large inductors that are used to enable the RF coil to be driven with DC. Specifically, they are placed in parallel to the tuning capacitors. Their self-resonance frequency is carefully tuned to 123 MHz (same as RF coil) so that they behave as an open circuit at such frequency, so they are virtually non-existent at the RF working frequency. The toroidal chokes depicted are selected to minimize the potential coupling among them and with the transmit field. FIG. 14C depicts a B0 shim-only coil 1405 added to the features in FIG. 4B.

FIG. 15 is a table that illustrates example results from Q factor measurements that indicate a MC coil 1405 contributes less than 25% of the noise at the RF loop 1404, according to an embodiment.

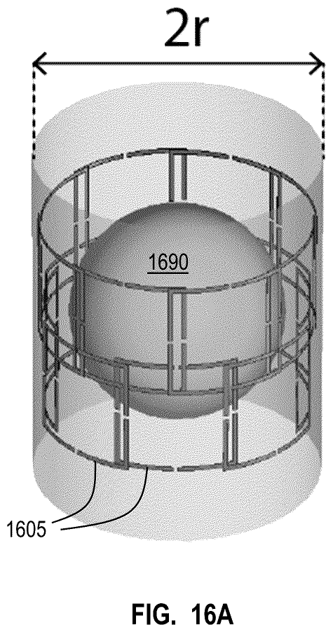

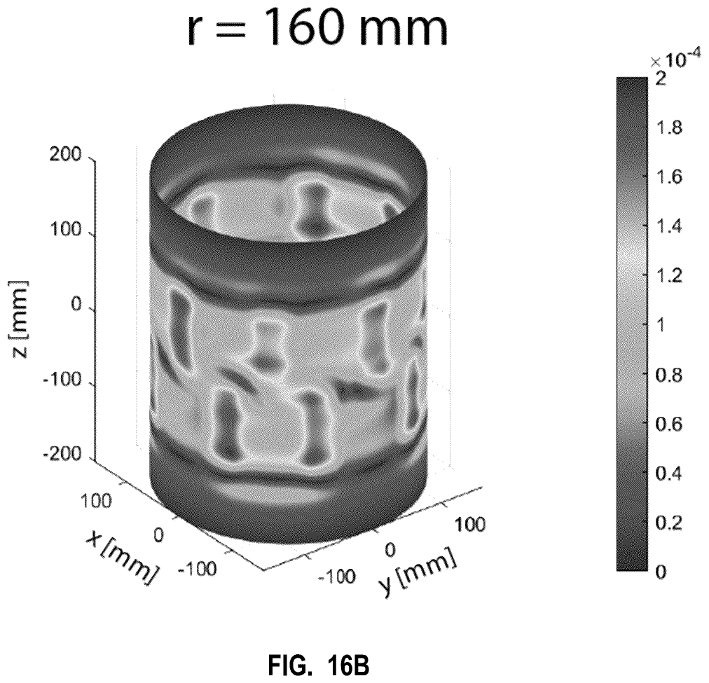

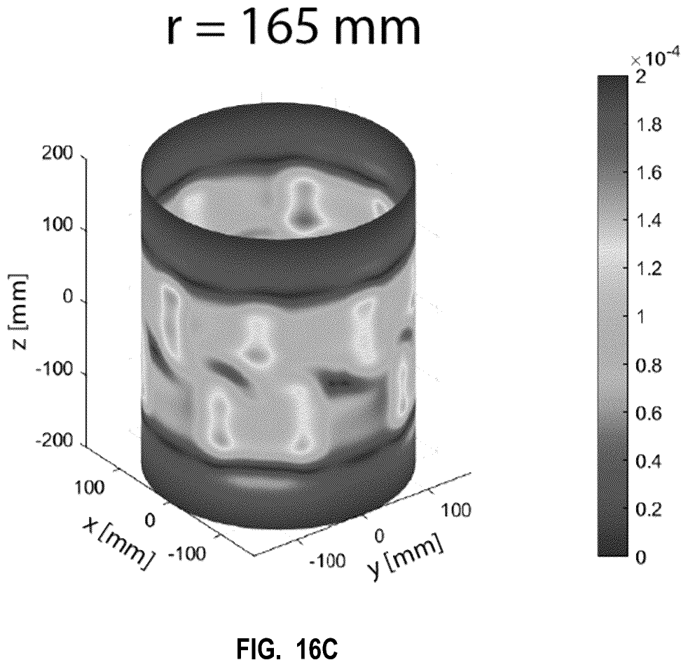

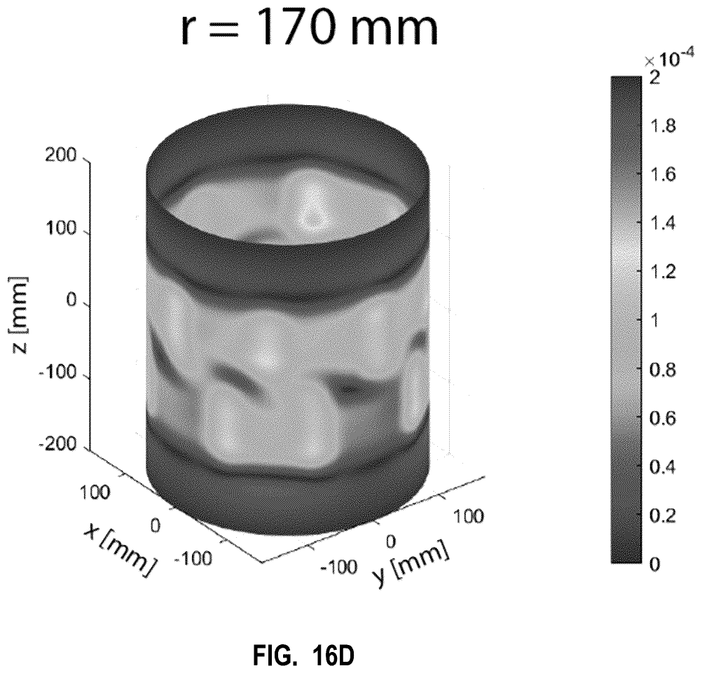

FIG. 16A through FIG. 16D are cylindrical maps that illustrate example magnetic flux obtained from sampling the array magnetic field flux through three cylindrical surfaces at different radii, according to an embodiment. FIG. 16A depicts the RF loops 1604 and phantom 1690 for a cylindrical measurement space of radius r. Each point represents the magnitude of the flux through a 40-turns 25-mm-radius B0 shim-only MC element centered in that location. FIG. 16B depicts flux at radius=160 mm. FIG. 16B depicts flux at radius=165 mm. FIG. 16C depicts flux at radius=170 mm. For |z|>125 mm, corresponding to the location of the B0 shim-only MC elements in the SNR experimental embodiment, a lower flux region is observed. The worst positioning is in correspondence of the vertical overlap regions among the RF elements. For this reason, the centers of the upper set of B0 shim-only coils 501 and the lower set were each placed apart from z_{L} in the B0 homogeneity experimental embodiments of FIG. 4B through FIG. 9E.

### 1.2 Magnetic Resonance Method

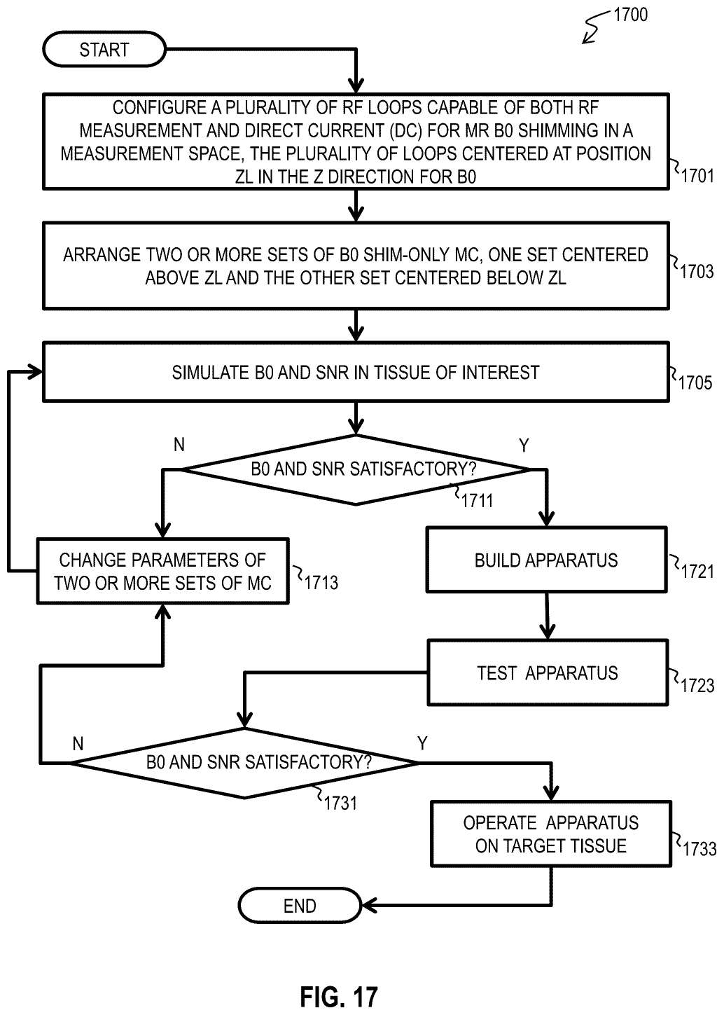

FIG. 17 is a method for configuring an apparatus with one or more RF loops and B0 shim-only MC coils, according to an embodiment. Although steps are depicted in FIG. 17 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways. In step 1701 a plurality of RF loops are configured to be capable of both RF measurement and direct current (DC) for magnetic resonance (MR) B0 shimming in a measurement space. The plurality of RF loops are centered in the z direction, designed for the direction of B0, at position z_{L}. The array of RF loops has diameter d_{L} in the z direction.

In step 1703, a candidate design is arranged with two or more sets of B0 shim-only MCs, at least one set centered in the z direction above z_{L} and at least one other set centered in the z direction below z_{L}. In step 1705, a simulation is run for the homogeneity of B0 in the measurement space and for the SNR in that space, as described in examples section.

In step 1711, it is determined whether the simulated B0 homogeneity and SNR is satisfactory for a particular purpose, e.g., to measure MR in the brain or heart of a simulated subject. In some embodiments, satisfactory is optimum in some sense, such as producing the smallest simulated signal to noise ratio in a region of interest among all configurations of the shim only coils tested, or a finite number of perturbations of those configurations. If not satisfactory, control passes to step 1713. In step 1713 the values of one or more parameters of the number and spacing and sizes of the B0 shim-only MCs are altered. Then control passes back to step 1705 to perform the simulations again with the new values of the number and spacing and size parameters. If it is determined in step 1711 that the B0 and SNR are satisfactory, then control passes to step 1721.

In step 1721m the apparatus as designed is fabricated, at least in test configuration. In step 1723, the apparatus is tested, e.g., on phantom or subjects with known attributes. The resulting B0 and SNR is measured.

In step 1731, it is determined whether the measured B0 homogeneity and SNR is satisfactory for a particular purpose, e.g., to measure MR in the brain or heart of a known subject. In some embodiments, satisfactory is optimum in some sense, such as producing the smallest measured signal to noise ratio in a region of interest among all configurations of the shim only coils tested, or a finite number of perturbations of those configurations. If not satisfactory, control passes back to step 1713 and following, described above, where the values of one or more parameters of the number and spacing and sizes of the B0 shim-only MCs are altered. If it is determined in step 1731 that the measured B0 and SNR are satisfactory, then control passes to step 1733.

In step 1733 the apparatus is operated for actual use on target tissue in each of one or more subjects.

### 2. Example Embodiments

In experimental embodiments, RF elements capable of running constant current (DC) are complemented with dedicated B0 shim-only MC elements. All of these elements are incorporated in one hybrid MR head coil for integration with a regular clinical MR system (specifically, a 3 T Siemens Prisma scanner). The design is such that improved B0 performance is achieved without degrading the MR signal strength, i.e. without decreasing sensitivity.

In the example embodiments, a cylindrical 16-channel RF head receive array was used with variously numbered and spaced MCs for shimming only. All RF and MC elements were assumed to be able to carry ±1 ampere (A) direct current (DC) for B0 shimming. The B0 shim-only MCs are sometimes referred to as MC-only elements. In some embodiments the rows of coils are called rings because the illustrated embodiment is a circular row.

In one set of simulations, the cylindrical 16-channel head receive array was the basis of this analysis. All RF+MC elements were assumed to be able to carry ±1 A direct current (DC) for B0 shimming. Five basic geometries with 2-6 rings (rows) with 8-16 MC elements (coils) each were created with varying coil diameter, distance between adjacent coils and the position of the rings along the z-axis and added to the 16 RF+MC elements. Dedicated (shim-only) MC coils were modeled as circular coils of 50 turns of wire each on a cylindrical surface carrying ±1 A maximum current. Coil fields in Hz/A for each of the total 400 discrete geometries were calculated using Biot-Savart simulations. The performance of each geometry was tested on 139 B field maps acquired in the adult human brain at 3 T (56±12 years, 87 women, 52 men). These maps were acquired as part of the Offspring Study in accordance with the Institutional Review Board of Columbia University. Regions of Interest (ROIs) of individual brain volumes were created using FSL's Brain extraction tool. Best MC fields were calculated for dynamic slice-by-slice shimming, and the standard deviation of the residual field across the ROIs was calculated. The average standard deviation over all subjects was used as a performance measure for a given MC geometry.

With a given number of coils, geometries with more rings performed slightly better than geometries with fewer rings. Optimal coil sizes depended on the positioning of the rings. Geometries with the coils distributed equally around the rings performed better than geometries with the coils aggregated towards the front of the setup.

In one experimental embodiment, there are 16 RF+MC elements (loops) and 18 MC-only elements (coils). A 3T 16-channel head receive array was simulated in CST Studio Suite (Dassault Systemes, France). The loops (138.5×135 mm) were symmetrically distributed around a cylindrical surface (radius: 151.5 mm) in two rows of 8 loops each. Each loop was tuned to 123 MHz, matched to 50Ω and geometrically decoupled to its nearest neighbors.

To quantify the effect of the presence of the MC-only coils on SNR, two configurations were simulated the 16-channel RF array (FIG. 4), and the 16-channel RF array of FIG. 4 with 18 MC-only shim coils distributed frontally above and below the RF coil (as shown in FIG. 7B). Signal to Noise Ratio (SNR) was calculated using Equation 1a and 1b.

$$S N R = \sqrt{{B}_{1}^{- *} {R}^{- 1} {B}_{1}^{-}} \qquad ( 1 a )$$

$$\begin{matrix}R = \int & σ {E}_{i} \cdot {E}_{j}^{*} d V\end{matrix} \qquad ( 1 b )$$

where R is the noise covariance matrix. The B0 field is the static magnetic field that is generated by the main magnet. The B1 field refers to the radio frequency magnetic field that is used to excite the protons (generated by the transmit RF coil) and the consequent field that they produce (picked up by the RF receive coil). Conventionally, we refer to B1+ as the transmit RF field and B1− as the receive RF field. R is the noise covariance matrix, and Eᵢ is the electric field produced by the i^{th} coil. The ‘*’ indicates the complex conjugate. RF+MC capability can be achieved with RF chokes across the tuning capacitors. The effect of the RF chokes, which were not included in the simulations, on the Q ratio of a single loop RF element was measured in three experimental configurations: (1) conventional RF-only as depicted in FIG. 14A, (2) RF+MC as depicted in FIG. 14B and (3) RF+MC with MC only coil in proximity as depicted in FIG. 14C. The RF chokes were hand-made by winding 59 turns of 22 AWG copper wire on a 3D printed toroidal support (OD/ID: 22/14 mm), as depicted in FIG. 14B, which resulted in an inductance L≈1.5 μH.

To guide the placement of the MC-only elements to minimize MC-to-RF coupling due to mutual inductance, the magnetic flux through three cylindrical surfaces with radii 160, 165, 170 mm was calculated. The flux on the cylindrical surfaces was sampled using a circular mask (radius: 25 mm, i.e. MC-only elements radius) and multiplied by 40 MC turns. For each surface, a flux map is obtained by assigning to each coordinate the magnitude of the flux through the circular mask centered in that location.

In another embodiment, a set of RF loops configured for a human head was used. A 16-channel receive (Rx) head helmet was built and tested at the bench. The array, constituted by 2 rows of 8 elements each, was built on a 3D-printed elliptical helmet former with diameters 24.5 cm (A-P) and 21 cm (R-L). FIG. 18A is a block diagram that illustrates an example of a phantom for a human head inside an array of RF loops, according to an embodiment. This embodiment comprises a 16-channel RF head array simulation setup. The 16 elements were distributed in two rows of 8 elements each. Nearest neighbors were decoupled via overlap, except for the two frontal elements of the bottom row, which were decoupled capacitively to allow enough space for two eye openings. FIG. 18B is a block diagram that illustrates the MR RF loops for a helmet-like measurement space, according to an embodiment. The elements were built with bus wire AWG 14 and tuned to 123 MHz with 5 series capacitors with values ranging from ˜20 pF to 24 pF. The coil was loaded with a phantom with dielectric properties εr=64 and σ=0.46 S/m (average of brain white and gray matter at 123 MHz).

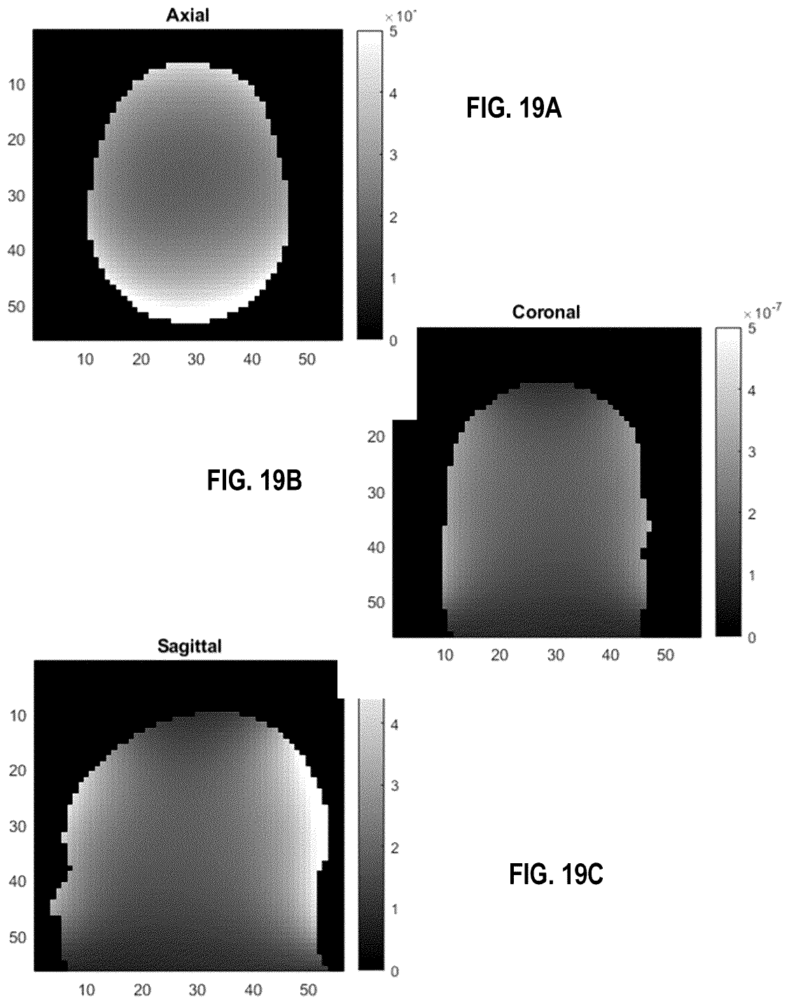

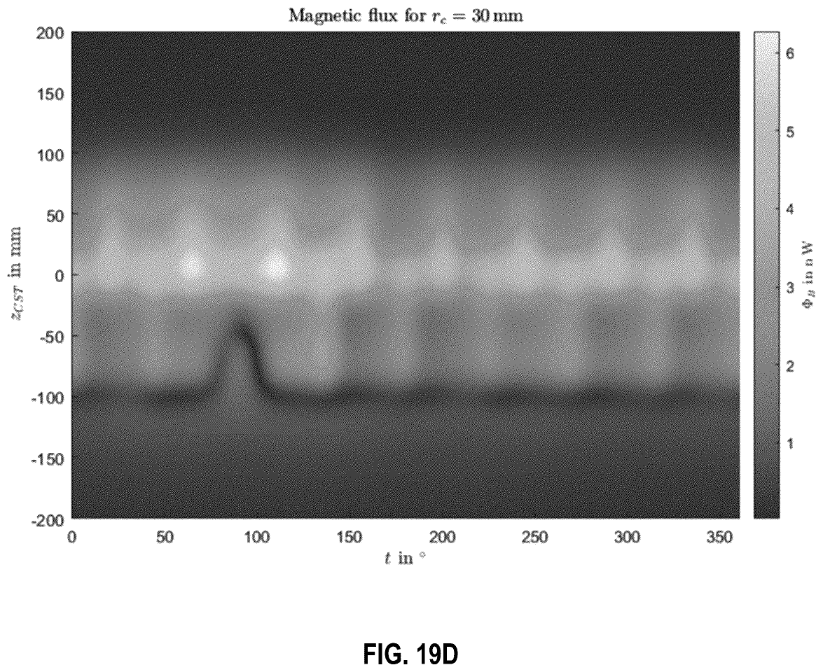

FIG. 19A, FIG. 19B and FIG. 19C are greyscale images that illustrate examples of simulated signal-to-noise ratio (SNR) in example axial, coronal and sagittal slices, respectively, from the RF coil setup illustrated in FIG. 18 before addition of MC shim only coils, according to an embodiment of the RF loops. FIG. 19D is an greyscale image of simulated magnetic flux generated by the 16-channel array through an elliptical cylinder distancing 30 mm from the RF coil. The flux map is shown flattened in 2D for simplicity. The horizontal axis represents the angular coordinate theta in degrees, the vertical axis represents the z coordinate in millimeters. High flux areas are indicated by the light areas and low flux by the dark areas.

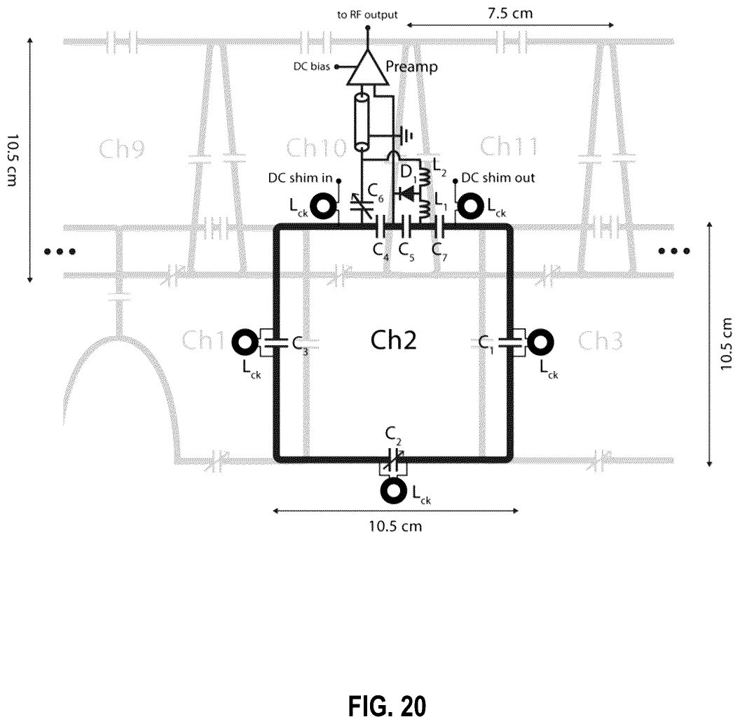

FIG. 20 is a block diagram that illustrates an example of a flattened 2D circuit schematic of an RF coil, according to an embodiment of the RF loops. For simplicity, a detailed diagram is shown for loop 2 (Ch2) of the array (in black), the rest of the elements are shown in light gray or omitted. The diode D1 allows detuning during RF transmission. Specifically, the status of the direct current bias is forward in transmit mode and reverse in receive mode. The RF chokes Lek provide a path for direct current allowing for B0 shimming operation. Typical component values are: C1=C2=C3=22 picoFarad (pF, 1 pF=10⁻¹² Farad), C4=C5=24 pF, C6=20 pF, C7=1000 pF, L1=68 nanoHenry (nH, 1 nH=10⁻⁹ Henry) L2=4.7 microHenry (μH, 1 μH=10⁻⁶ Henry), Lck=1.5 μH. Each RF loop is formed as a square. Each of the capacitors C1-C5 is in parallel to inductor Lck. Inductors combined with capacitors can be used to create tuned LC circuits. The measured Q ratio is given by Equation 2.

$${Q}_{ratio} = \frac{{Q}_{u n l o a d e d}}{{Q}_{l o a d e d}} = \frac{331.3 \pm 23.4}{100.4 \pm 19.3} = 3.4 \pm 0.7 \qquad ( 2 )$$

High input impedance preamplifiers provide <−31 dB further decoupling (i.e., provide preamp decoupling) among non-neighboring elements of the array. The preamplifier is labeled in FIG. 20 as Preamp. During transmission (Tx), the Rx array is detuned with one active high impedance circuit at each port, providing <−19 dB detuning. A 4 cm-long coaxial cable may be used to connect the output of the RF coil to the preamp.

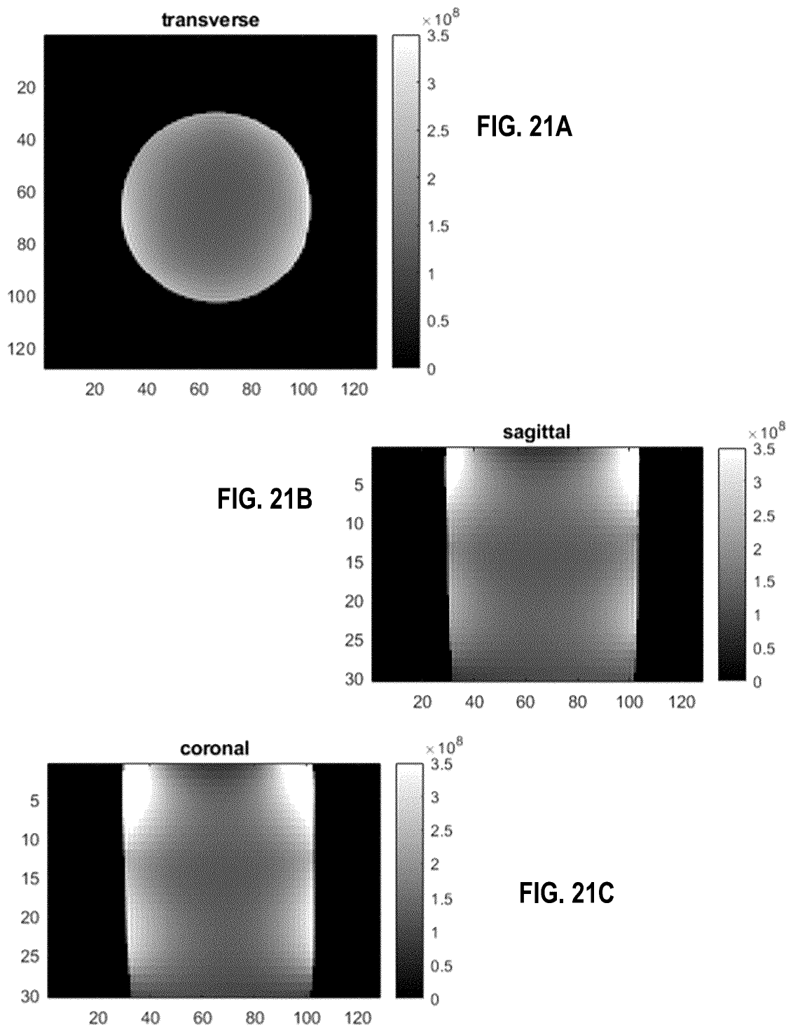

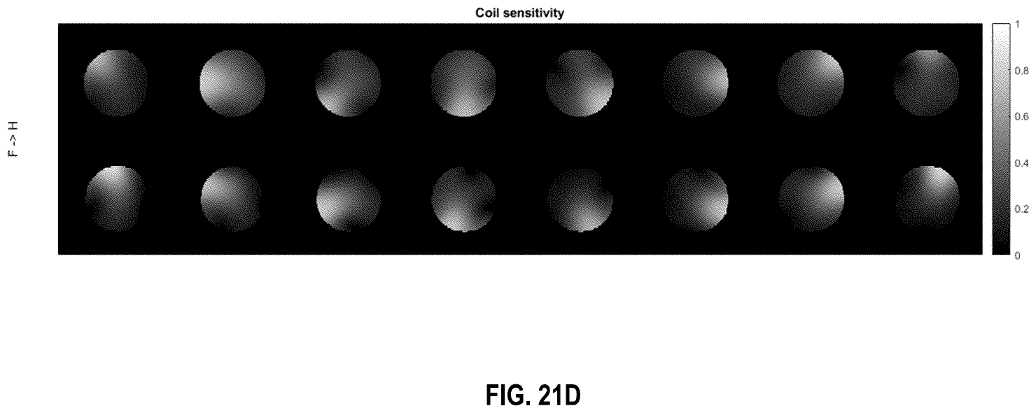

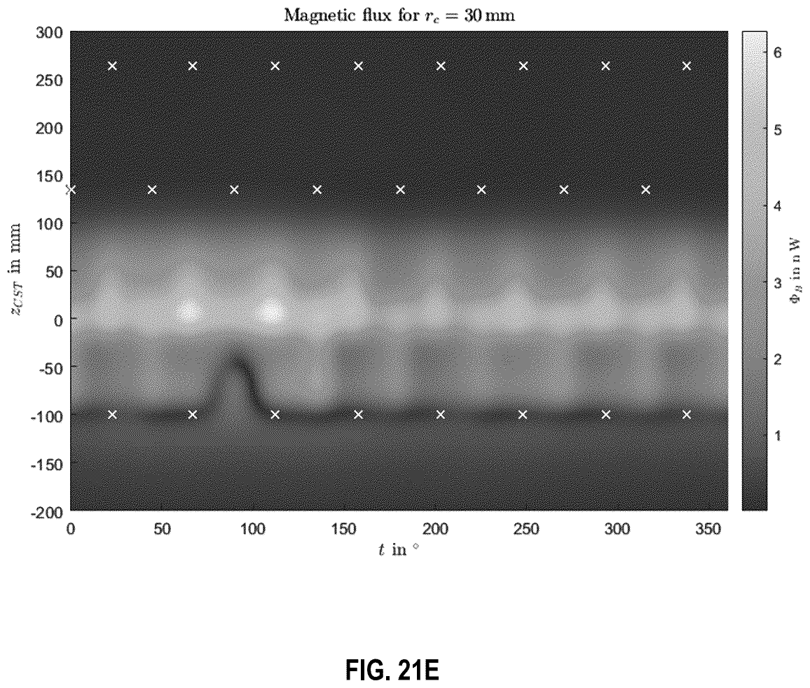

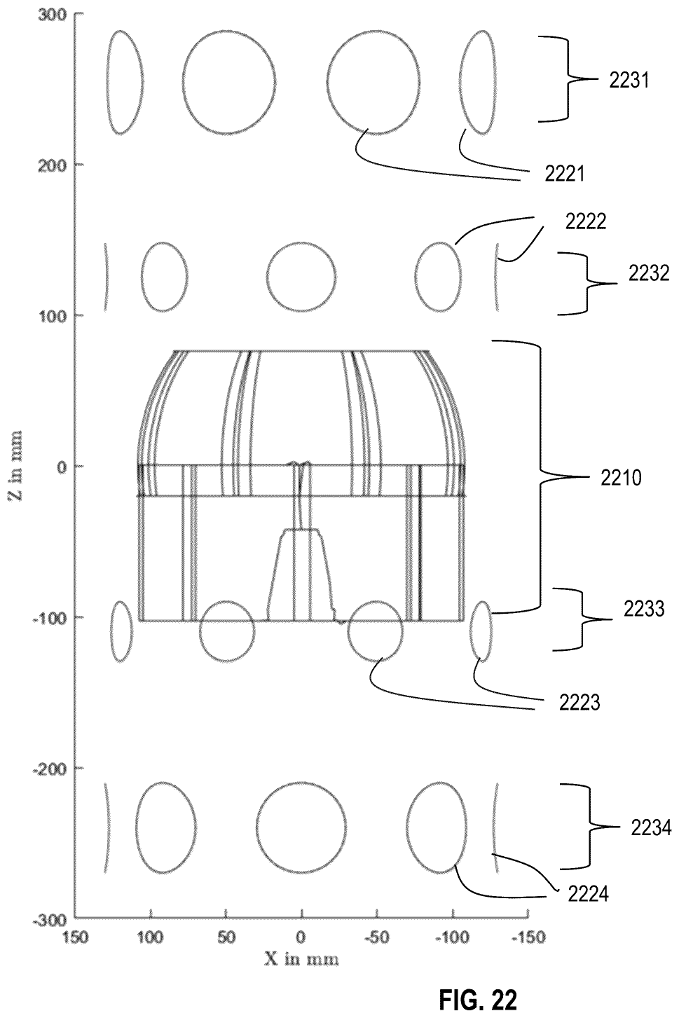

FIG. 21A, FIG. 21B and FIG. 21C are greyscale images that illustrate examples of transverse, sagittal and coronal slices, respectively, of measured SNR of the 16-channel RF array before inserting the RF chokes or the MC shim-only coils, according to an embodiment of the RF loops. It is advantageous for this signal to not be degraded substantially by adding the MC shim-only coils. FIG. 21D is a set of greyscale images that illustrate examples of measured coil sensitivity for each loop of the RF array in its RF-only configuration, according to this embodiment of the RF loops.

To find optimum placement for MC-only elements, MC-only elements were added to the 16-channel dome-shaped RF channels (1 turn, 1 A max). Based on the preliminary analysis described above, the number of MC-only elements was limited to 32, four rows of eight. The size and location of these elements was numerically optimized utilizing a set of 139 in vivo full brain B0 maps (56+/−12 years, 88f/51 m). The optimization was performed by minimizing the average standard deviation of the residual B0 field in the brain over all in vivo cases. To minimize coupling between the RF elements and the MC-only elements, the total magnetic flux through these coils was calculated in each step. To improve runtime, computationally expensive simulations such as Biot-Savart simulations of coil fields and the calculation of the magnetic flux through a coil were performed beforehand for a range of coil positions and sizes. During the simulation in step 1705, these values were interpolated from this existing data.

The most advantageous configuration among those simulated using these constraints is shown below and includes 4 rows of 8 elements each, placed at the z locations z=−240, −110, 120, 190 mm. The MC element radii are r=30, 20, 26, 34 mm for rows 1 through 4, respectively. FIG. 21E is plot showing example locations for MC coils superposed on the greyscale image of the simulated magnetic flux of FIG. 19D, according to an embodiment. The white crosses correspond to the center of the MC elements, whose location was optimized for optimal B0 shimming and minimal coupling to the RF coil. This can be done by determining that interference with a received RF signal in the one or more RF loops is below a target level of interference. SNR thresholds can vary from application to application but 1%, 2% and 5% are acceptable. As can be seen these locations correspond to areas of low simulated magnetic flux from the RF loops. Thus, in some embodiments, the MC shim-only coils are centered on a region where magnetic flux from the RF loops is below a magnetic flux threshold. For example a magnetic flux threshold can be expressed as a enumerated magnetic flux at a certain radius (e.g., a radius of 30 mm) or as a value that is a certain percentile of the simulated fluxes at that radius, e.g., the 1^{st} percentile or the 5th percentile or the 10th percentile of fluxes simulated at that radius. Thus, interference with the received RF signal in the one or more RF loops is below the target level of interference when the plurality of coils configured only for B0 shimming in the measurement space are spaced to be centered on a region where magnetic flux from the RF loops is below a threshold. Thus, in some embodiments, the magnetic flux threshold is a value of magnetic flux below a percentile selected in a range from a 1st percentile to a 10th percentile of fluxes simulated at a predetermined radius. Alternatively, flux values below a percentage of the maximum total flux, in ranges from 1% to 5%, may be used.

FIG. 22 is a block diagram that illustrates an example schematic representation of MC coils (4 rows of 8 circular elements) surrounding the RF coil (2 rows of 8 squared loops, appropriately bent to follow the contour of a head helmet), according to an embodiment. The performance of this arrangement is depicted in FIG. 10 as the case labeled MC 4×8 on the horizontal axis. Shown is the average shim performance σ(ΔB) and its standard deviation across all 139 in vivo subjects. The average shim performance with this geometry was 10.6 Hz. For this advantageous geometry, the addition of the B0 shim-only MC outperforms spherical harmonics currently available clinically.

For testing purposes, to demonstrate by measurements the predictions made by simulation, a mockup panel was built with 5 MC elements. B0 field maps with the test mockup were acquired and are shown below.

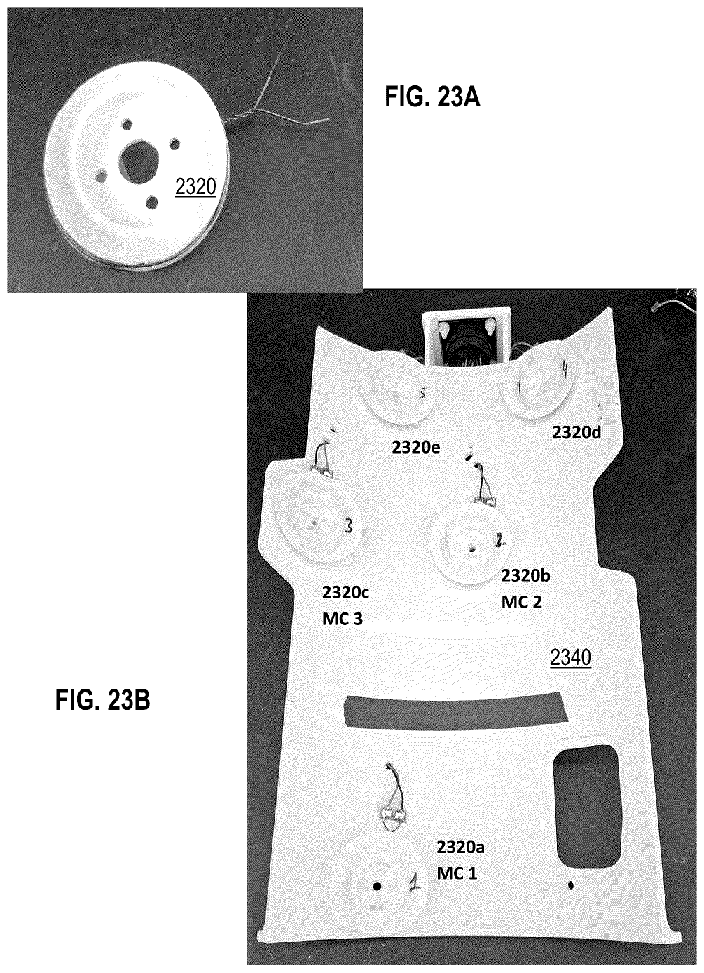

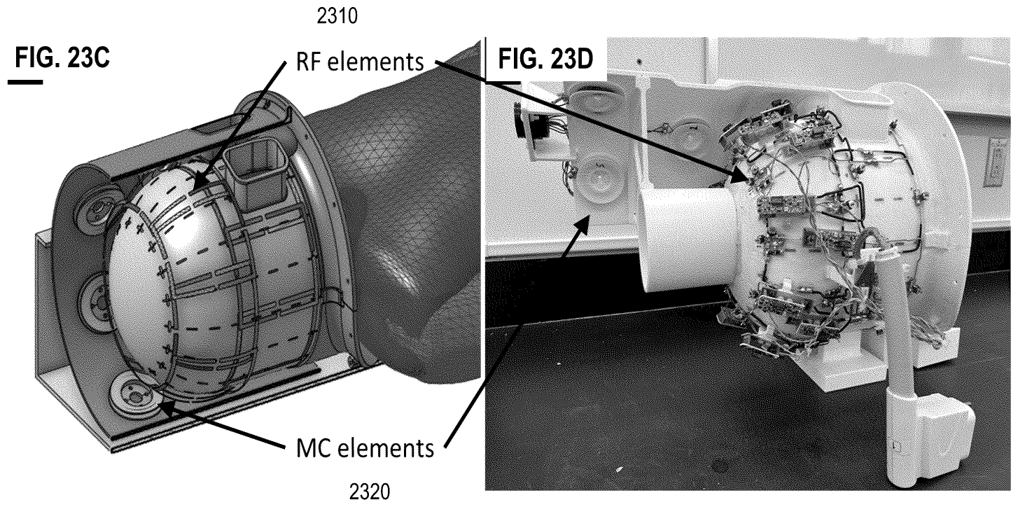

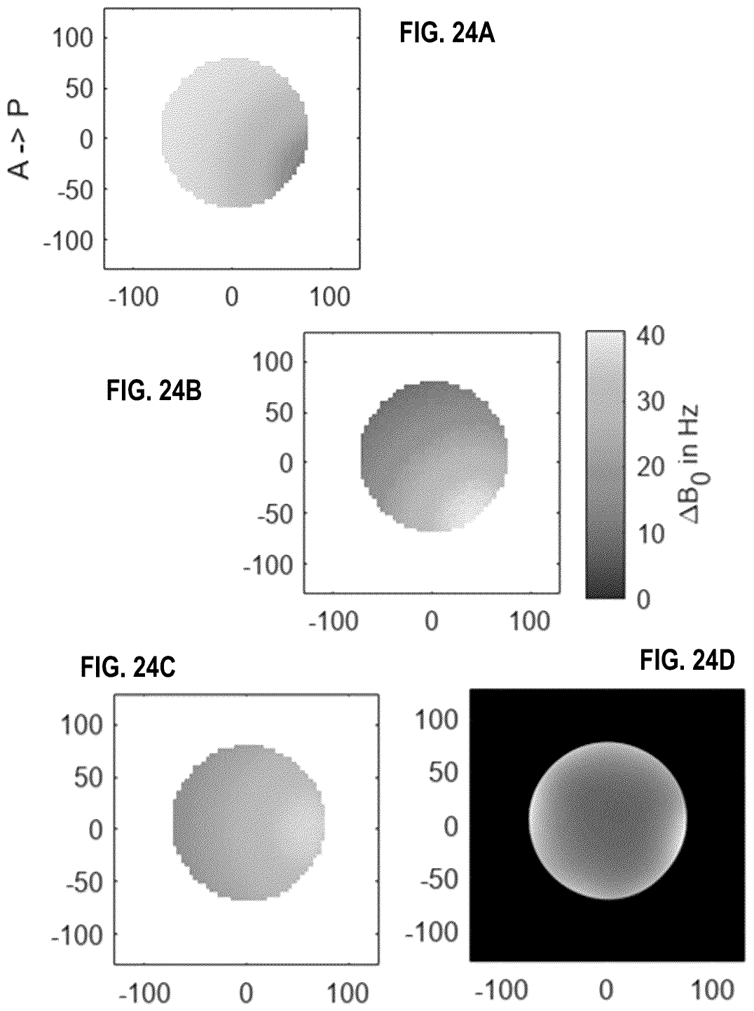

The MC elements (coils) were 50 mm diameter bundle inductors built with 40 turns of 20 AWG magnet wire. Each element was built on a 3D printed form with holes for mounting onto an elliptical cylinder surrounding the RF elements (loops). FIG. 23A is a photograph that depicts an example of a multicoil shim-only coil element 2320, according to an embodiment. FIG. 23B is a photograph that depicts an example panel 2340 of multiple shim-only coils 2320, according to an embodiment. Five MC shim-only coils 2320a, 2320b, 2320c, 2320d and 2320e (collectively referenced as MC shim-only coils 2320) are depicted. The thick grey strip on the panel 2340 indicates the z-coordinate of the center of the RF loop helmet. FIG. 23C is a block diagram that illustrates the experimental panel 2340 with MC shim-only coils 2320 placed adjacent a helmet of RF elements (loops) 2310, according to an embodiment. FIG. 23D is a photograph that illustrates the experimental panel 2340 with MC shim-only coils 2320 placed adjacent a helmet of RF elements (loops) 2310, according to an embodiment. In this experiment, only B0 maps were looked examined. FIG. 24A, FIG. 24B and FIG. 24 C are greyscale images that illustrated examples of B0 field maps produced by MC shim-only coils 2320a, 2320b and 2320c, respectively, depicted in FIG. 23B, according to an embodiment; and FIG. 24 D is a high resolution MR image of the spherical phantom used for the experiment, according to an embodiment.

### 3. Computational Hardware Overview

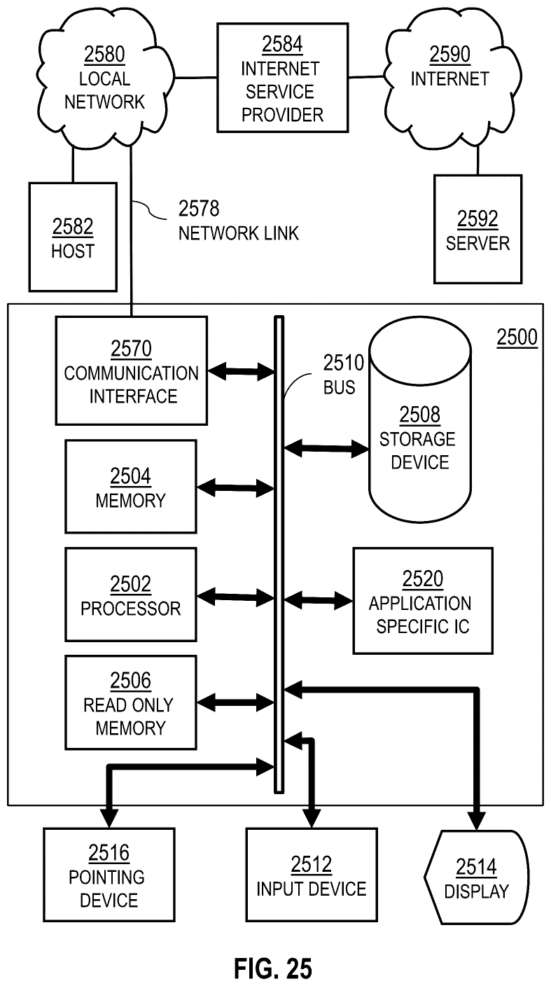

FIG. 25 is a block diagram that illustrates a computer system 2500 upon which an embodiment of the invention may be implemented. Computer system 2500 includes a communication mechanism such as a bus 2510 for passing information between other internal and external components of the computer system 2500. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 2500, or a portion thereof, constitutes a means for performing one or more steps of one or more methods described herein.

A sequence of binary digits constitutes digital data that is used to represent a number or code for a character. A bus 2510 includes many parallel conductors of information so that information is transferred quickly among devices coupled to the bus 2510. One or more processors 2502 for processing information are coupled with the bus 2510. A processor 2502 performs a set of operations on information. The set of operations include bringing information in from the bus 2510 and placing information on the bus 2510. The set of operations also typically includes comparing two or more units of information, shifting positions of units of information, and combining two or more units of information, such as by addition or multiplication. A sequence of operations to be executed by the processor 2502 constitutes computer instructions.

Computer system 2500 also includes a memory 2504 coupled to bus 2510. The memory 2504, such as a random access memory (RAM) or other dynamic storage device, stores information including computer instructions. Dynamic memory allows information stored therein to be changed by the computer system 2500. RAM allows a unit of information stored at a location called a memory address to be stored and retrieved independently of information at neighboring addresses. The memory 2504 is also used by the processor 2502 to store temporary values during execution of computer instructions. The computer system 2500 also includes a read only memory (ROM) 2506 or other static storage device coupled to the bus 2510 for storing static information, including instructions, that is not changed by the computer system 2500. Also coupled to bus 2510 is a non-volatile (persistent) storage device 2508, such as a magnetic disk or optical disk, for storing information, including instructions, that persists even when the computer system 2500 is turned off or otherwise loses power.

Information, including instructions, is provided to the bus 2510 for use by the processor from an external input device 2512, such as a keyboard containing alphanumeric keys operated by a human user, or a sensor. A sensor detects conditions in its vicinity and transforms those detections into signals compatible with the signals used to represent information in computer system 2500. Other external devices coupled to bus 2510, used primarily for interacting with humans, include a display device 2514, such as a cathode ray tube (CRT) or a liquid crystal display (LCD), for presenting images, and a pointing device 2516, such as a mouse or a trackball or cursor direction keys, for controlling a position of a small cursor image presented on the display 2514 and issuing commands associated with graphical elements presented on the display 2514.

In the illustrated embodiment, special purpose hardware, such as an application specific integrated circuit (IC) 2520, is coupled to bus 2510. The special purpose hardware is configured to perform operations not performed by processor 2502 quickly enough for special purposes. Examples of application specific ICs include graphics accelerator cards for generating images for display 2514, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 2500 also includes one or more instances of a communications interface 2570 coupled to bus 2510. Communication interface 2570 provides a two-way communication coupling to a variety of external devices that operate with their own processors, such as printers, scanners and external disks. In general, the coupling is with a network link 2578 that is connected to a local network 2580 to which a variety of external devices with their own processors are connected. For example, communication interface 2570 may be a parallel port or a serial port or a universal serial bus (USB) port on a personal computer. In some embodiments, communications interface 2570 is an integrated services digital network (ISDN) card or a digital subscriber line (DSL) card or a telephone modem that provides an information communication connection to a corresponding type of telephone line. In some embodiments, a communication interface 2570 is a cable modem that converts signals on bus 2510 into signals for a communication connection over a coaxial cable or into optical signals for a communication connection over a fiber optic cable. As another example, communications interface 2570 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 2570 sends and receives electrical, acoustic or electromagnetic signals, including infrared and optical signals, that carry information streams, such as digital data.

The term computer-readable medium is used herein to refer to any medium that participates in providing information to processor 2502, including instructions for execution. Such a medium may take many forms, including, but not limited to, non-volatile media, volatile media and transmission media. Non-volatile media include, for example, optical or magnetic disks, such as storage device 2508. Volatile media include, for example, dynamic memory 2504. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 2502, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 2502, except for carrier waves and other signals.

Logic encoded in one or more tangible media includes one or both of processor instructions on a computer-readable storage media and special purpose hardware, such as ASIC 2520.

Network link 2578 typically provides information communication through one or more networks to other devices that use or process the information. For example, network link 2578 may provide a connection through local network 2580 to a host computer 2582 or to equipment 2584 operated by an Internet Service Provider (ISP). ISP equipment 2584 in turn provides data communication services through the public, world-wide packet-switching communication network of networks now commonly referred to as the Internet 2590. A computer called a server 2592 connected to the Internet provides a service in response to information received over the Internet. For example, server 2592 provides information representing video data for presentation at display 2514.

The invention is related to the use of computer system 2500 for implementing the techniques described herein. According to one embodiment of the invention, those techniques are performed by computer system 2500 in response to processor 2502 executing one or more sequences of one or more instructions contained in memory 2504. Such instructions, also called software and program code, may be read into memory 2504 from another computer-readable medium such as storage device 2508. Execution of the sequences of instructions contained in memory 2504 causes processor 2502 to perform the method steps described herein. In alternative embodiments, hardware, such as application specific integrated circuit 2520, may be used in place of or in combination with software to implement the invention. Thus, embodiments of the invention are not limited to any specific combination of hardware and software.

The signals transmitted over network link 2578 and other networks through communications interface 2570, carry information to and from computer system 2500. Computer system 2500 can send and receive information, including program code, through the networks 2580, 2590 among others, through network link 2578 and communications interface 2570. In an example using the Internet 2590, a server 2592 transmits program code for a particular application, requested by a message sent from computer 2500, through Internet 2590, ISP equipment 2584, local network 2580 and communications interface 2570. The received code may be executed by processor 2502 as it is received, or may be stored in storage device 2508 or other non-volatile storage for later execution, or both. In this manner, computer system 2500 may obtain application program code in the form of a signal on a carrier wave.

Various forms of computer readable media may be involved in carrying one or more sequence of instructions or data or both to processor 2502 for execution. For example, instructions and data may initially be carried on a magnetic disk of a remote computer such as host 2582. The remote computer loads the instructions and data into its dynamic memory and sends the instructions and data over a telephone line using a modem. A modem local to the computer system 2500 receives the instructions and data on a telephone line and uses an infra-red transmitter to convert the instructions and data to a signal on an infra-red a carrier wave serving as the network link 2578. An infrared detector serving as communications interface 2570 receives the instructions and data carried in the infrared signal and places information representing the instructions and data onto bus 2510. Bus 2510 carries the information to memory 2504 from which processor 2502 retrieves and executes the instructions using some of the data sent with the instructions. The instructions and data received in memory 2504 may optionally be stored on storage device 2508, either before or after execution by the processor 2502.

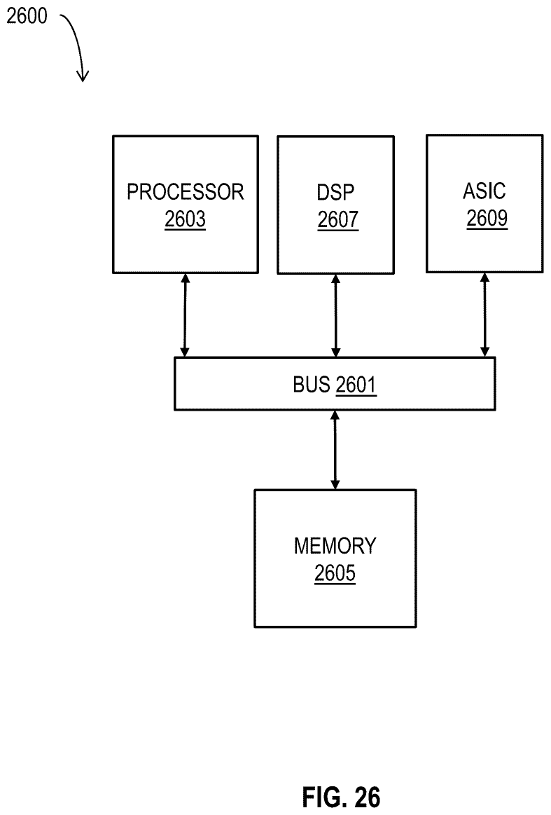

FIG. 26 illustrates a chip set 2600 upon which an embodiment of the invention may be implemented. Chip set 2600 is programmed to perform one or more steps of a method described herein and includes, for instance, the processor and memory components described with respect to FIG. 25 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 2600, or a portion thereof, constitutes a means for performing one or more steps of a method described herein.

In one embodiment, the chip set 2600 includes a communication mechanism such as a bus 2601 for passing information among the components of the chip set 2600. A processor 2603 has connectivity to the bus 2601 to execute instructions and process information stored in, for example, a memory 2605. The processor 2603 may include one or more processing cores with each core configured to perform independently. A multi-core processor enables multiprocessing within a single physical package. Examples of a multi-core processor include two, four, eight, or greater numbers of processing cores. Alternatively, or in addition, the processor 2603 may include one or more microprocessors configured in tandem via the bus 2601 to enable independent execution of instructions, pipelining, and multithreading. The processor 2603 may also be accompanied with one or more specialized components to perform certain processing functions and tasks such as one or more digital signal processors (DSP) 2607, or one or more application-specific integrated circuits (ASIC) 2609. A DSP 2607 typically is configured to process real-world signals (e.g., sound) in real time independently of the processor 2603. Similarly, an ASIC 2609 can be configured to perform specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 2603 and accompanying components have connectivity to the memory 2605 via the bus 2601. The memory 2605 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 2605 also stores the data associated with or generated by the execution of one or more steps of the methods described herein.

### 4. Alterations, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### 5. References

The reference cited below are hereby each incorporated by reference as if fully set forth herein, except for terminology inconsistent with that used herein.
Juchem, C, et al. (2011), Dynamic multi-coil shimming of the human brain at 7 T. Journal of Magnetic Resonance 212(2), 280-288.
Stockmann, J. P., et al. (2016), A 32-channel combined RF and B0 shim array for 3T brain imaging. Magnetic Resonance in Medicine, 75(1), 441-451).
Juchem, C., et al. (2021), Magnetic Resonance Imager with Coils for Arbitrary Image Space and Fabrication Thereof. Patent No. WO2021/022252).
Juchem C, Brown P B, Nixon T W, et al. Multicoil shimming of the mouse brain. Magn Reson Med. 2011; 66(3):893-900.
Juchem C, Herman P, Sanganahalli B, et al. DYNAmic Multi-coIl TEchnique (DYNAMITE) shimming of the rat brain at 11.7T. NMR in Biomedicine, 2014; 27(8):897-906.
Darnell D, Ma Y, Wang H, et al. Adaptive integrated parallel reception, excitation, and shimming (iPRES-A) with microelectromechanical systems switches. Magn Reson Med. 2018; 80(1):371-379.
Juchem C. (2017). BODETOX—B0 Detoxication Software for Magnetic Field Shimming. Columbia University Technology Ventures. http://innovation.columbia.edu/technologies/cul7326_bOdetox
Manly J, Renteria M A, Avila-Rieger J F, et al. Offspring Study of Racial and Ethnic Disparities in Alzheimer's Disease: Objectives and Design. PsyArXiv. 2020. Preprint. https://doi.org/10.31234/OSF.IO/FRBKJ
Jenkinson M, Beckmann C F, Behrens T E J, et al. FSL. Neurolmage. 2012; 62(2):782-790.
Smith S M. Fast robust automated brain extraction. Human Brain Mapping. 2002; 17(3):143-155.
de Graaf R A, Juchem C. CHAPTER 4. B0 Shimming Technology. In Magnetic Resonance Technology: Hardware and System Component Design. The Royal Society of Chemistry. 2016; 166-207.
Ianniello C, Theilenberg S, Majumder J A, et al. A 16-channel MC and RF hybrid system for B0 shimming and B1 reception: a preliminary prototype. Proc Intl Soc Magn Reson Med. 2023, Toronto, Canada. Under review.
Juchem, C.; Nixon, T. W.; McIntyre, S.; Rothman, D. L.; de Graaf, R. A., Magnetic field modeling with a set of individual localized coils. J Magn Reson 2010,204 (2), 281-9.
Juchem, C.; Umesh Rudrapatna, S.; Nixon, T. W.; de Graaf, R. A., Dynamic multi-coil technique (DYNAMITE) shimming for echo-planar imaging of the human brain at 7 Tesla. Neuroimage 2015, 105, 462-72.
Roemer, P. B.; Edelstein, W. A.; Hayes, C. E.; Souza, S. P.; Mueller, O. M., The NMR phased array. Magn. Reson. Med. 1990, 16 (2), 192-225.
Theilenberg S.; Ianniello, C.; Juchem, C., Design of a Hybrid Multi-Coil Array for B0 Shimming of the In Vivo Human Brain, Proc ISMRM, 2023, Toronto, Canada, under review.
Gabriel, S.; Lau, R. W.; Gabriel, C., The dielectric properties of biological tissues: II. Measurements in the frequency range 10 Hz to 20 GHz. Phys Med Biol 1996, 41 (11), 2251-69.

What is claimed is:

1. An apparatus for magnetic resonance measurements, the apparatus comprising:
one or more radio-frequency (RF) loops configured to probe a measurement space; and
a plurality of coils configured only for B0 shimming in the measurement space and spaced such that interference with a received RF signal in the one or more RF loops is below a target level of interference.

2. The apparatus as recited in claim 1, wherein:
B0 is directed in a z direction;
N is a number of rows in the z direction of the one or more RF loops;
the one or more RF loops are centered at z position z_{L};
each loop has a diameter no smaller than d_{L};
each coil of the plurality of coils has a diameter no larger than a maximum diameter d_{C} that is smaller than N d_{L}
the plurality of coils is arranged in at least two sets of rows, a first set centered at a z position z1 less than z_{L} and a second set centered at a z position z2 greater than z_{L}, each set of rows on a periphery of the measurement space;
a distance [symbol] z=z2−z1; and
$${d}_{C} < ⊗ z < {Nd}_{L} .$$

3. The apparatus as recited in claim 1, wherein the measurement space is a cylinder.

4. The apparatus as recited in claim 1, wherein each loop is configured to carry up to 1 ampere of current independently of every other loop and each coil of the plurality of coils is configured to carry at least the current of each loop.

5. The apparatus as recited in claim 1, wherein each loop has only one winding of an insulated conductor and each coil of the plurality of coils has at least 10 windings of an insulated conductor.

6. The apparatus as recited in claim 1, wherein interference with the received RF signal in the one or more RF loops is below the target level of interference when the plurality of coils configured only for B0 shimming in the measurement space are spaced to be centered on a region where magnetic flux from the RF loops is below a threshold.

6. The apparatus as recited in claim 1, wherein the magnetic flux threshold is a value of magnetic flux below a percentile selected in a range from a 1^{st} percentile to a 10th percentile of fluxes simulated at a predetermined radius.

7. A system for magnetic resonance measurements comprising.
the apparatus of claim 1;
a plurality of gradient field coils; and
a controller configured to operate the gradient field coils and the apparatus.
